(19) United States
(12) Patent Application Publication
(10) Pub. No.: US 2024/0301085 A1
(43) Pub. Date: Sep. 12, 2024

(54) HUMANIZED GPC3 ANTIBODY AND APPLICATION THEREOF

(71) Applicant: SIMCERE ZAIMING PHARMACEUTICAL CO., LTD., Haikou (CN)

(72) Inventors: Hu GE, Shanghai (CN); Shumei YOU, Shanghai (CN)

(73) Assignee: SIMCERE ZAIMING PHARMACEUTICAL CO., LTD., Haikou (CN)

(21) Appl. No.: 18/275,901

(22) PCT Filed: Feb. 9, 2022
(86) PCT No.: PCT/CN2022/075588

§ 371 Date: Aug. 4, 2023

(30) Foreign Application Priority Data
Feb. 10, 2021 (CN) 202110182284.0

(51) Int. Cl. C07K 16/30; A61K 39/00; G01N 33/68

(52) U.S. Cl. CPC C07K 16/303; A61K 39/4631; G01N 33/6854; A61K 2039/505; A61K 2239/13; A61K 2239/21; A61K 2239/22; C07K 2317/24; C07K 2317/31; C07K 2317/565; C07K 2317/567; C07K 2317/92

(57) ABSTRACT

Provided is a humanized antibody or antigen-binding fragment that specifically binds to GPC3. The antibody or antigen-binding fragment has high affinity with GPC3 protein, and can be used in the preparation of drugs for treating tumors and the like. Also provided are a nucleic acid molecule encoding the humanized GPC3 antibody or antigen-binding fragment, an expression vector, a host cell, and a method for preparing the antibody or antigen-binding fragment. Also provided are an immunoconjugate comprising same, a chimeric antigen receptor, an immunocompetent cell, a multispecific molecule, and a pharmaceutical composition. Also provided are a method for detecting GPC3, and a method for treating various GPC3-related disorders including hepatocellular carcinoma.

The present application claims priority to the Chinese Patent Application No. 202110182284.0 entitled “HUMANIZED GPC3 ANTIBODY AND APPLICATION THEREOF” and filed with the China National Intellectual Property Administration on Feb. 10, 2021, which is incorporated herein by reference in its entirety.

### TECHNICAL FIELD

The present invention relates to the field of antibodies, in particular to a GPC3 humanized antibody and use thereof.

### BACKGROUND

Glypican-3 (GPC3) is a heparan sulfate (HS) glycoprotein, belonging to a family of the heparan sulfate proteoglycans. GPC3 is attached to the cell membrane by a glycophosphatidylinositol (GPI) anchor. The GPC3 core protein consists of 580 amino acids and is about 70 kD in size. The protein can be cleaved by furin to produce a 40-kD N-terminal subunit and a 30-kD C-terminal subunit, and the two subunits are linked by disulfide bonds. Two HS side chains of GPC3 are attached near the C-terminal position (Takahiro Nishida, Hiroaki Kataoka. Glypican 3-Targeted Therapy in Hepatocellular Carcinoma, Cancers 2019; 11(9):1339).

GPC3 is believed to play a crucial regulatory role in cellular proliferation in embryonic mesodermal tissues since deletion of the GPC3 gene leads to the development of gigantism/overgrowth syndrome known as Simpson-Golabi-Behmel syndrome (SGBS). GPC3 is expressed throughout the fetal period, but is seldom expressed after birth in normal tissues despite the weak expression in placental, mammary, mesothelial, ovarian, lung and kidney tissues. Abnormal expression of GPC3 has been reported in various tumor tissues in adults, such as hepatocellular carcinoma (HCC), lung squamous cell carcinoma, gastric cancer, and ovarian cancer. GPC3 is highly expressed especially in HCC cells, and promotes growth and invasion of HCC cells by enhancing autocrine/paracrine canonical Wnt signaling (Capurro M I, Xiang Y-Y, Lobe C, Filmus J. Glypican-3 promotes the growth of hepatocellular carcinoma by stimulating canonical Wnt signaling. Cancer Res 2005; 65:6245-54). Immunohistochemical staining revealed that high expression of GPC3 protein was observed in tumor tissues of approximately 70% of patients with HCC (Capurro M, Wanless I R, Sherman M, et al. Glypican-3: a novel serum and histochemical marker for hepatocellular carcinoma. Gastroenterology 2003; 125:89-97), and therefore, GPC3 is considered a candidate target for tumor therapy.

As a recombinant humanized monoclonal antibody developed by Chugai Pharmaceutical, codrituzumab (also known as GC33 antibody) binds to a membrane proximal region of the GPC3 protein. The GC33 antibody targets GPC3-positive HCC cells and can produce antibody-dependent cellular cytotoxicity (ADCC). In a clinical phase I trial, codrituzumab showed good immune tolerance and produced anti-tumor effects in patients with HCC (Ikeda M, Ohkawa S, Okusaka T, et al. Japanese phase I study of GC33, a humanized antibody against glypican-3 for advanced hepatocellular carcinoma. Cancer Sci. 2014, 105, 455-462). However, in a phase II clinical trial that enrolled 185 patients with advanced hepatocellular carcinoma, codrituzumab was less effective compared with the control, and investigators concluded that patient outcomes could be improved either by using high doses of codrituzumab or by selecting patients expressing higher levels of GPC3 or CD16 (Abou-Alfa G. K, Puig O, Daniele B, et al. Randomized phase II placebo controlled study of codrituzumab in previously treated patients with advanced hepatocellular carcinoma. J. Hepatol. 2016, 65, 289-295). In conclusion, clinical application of this antibody remains to be discussed.

### SUMMARY

In response to the problems in the prior art, the present invention provides a humanized antibody or an antigen-binding fragment specifically binding to GPC3. The present invention further provides an immunoconjugate comprising the antibody or the antigen-binding fragment, a chimeric antigen receptor, an immunocompetent cell, a multispecific molecule, a nucleic acid molecule, an expression vector, a host cell, a pharmaceutical composition, a preparation method, and use.

According to a first aspect, the present invention provides an antibody or an antigen-binding fragment specifically binding to GPC3, comprising a humanized heavy chain variable region and/or a humanized light chain variable region, wherein the heavy chain variable region comprises a human-derived heavy chain framework region, a complementarity determining region (CDR) 1 comprising an amino acid sequence set forth in SEQ ID NO: 15, a CDR2 comprising an amino acid sequence set forth in SEQ ID NO: 16, and a CDR3 comprising an amino acid sequence set forth in SEQ ID NO: 17; and the light chain variable region comprises a human-derived light chain framework region, a complementarity determining region (CDR) 1 comprising amino acid sequences set forth in SEQ ID NOs: 18 and 21, a CDR2 comprising an amino acid sequence set forth in SEQ ID NO: 19, and a CDR3 comprising an amino acid sequence set forth in SEQ ID NO: 20.

In some embodiments, the heavy chain framework region comprises framework regions HFR1, HFR2 and HFR3 of IGHV1-3*01 set forth in SEQ ID NO: 11, and a framework region HFR4 of IGHJ1*01 set forth in SEQ ID NO: 12; and the light chain framework region comprises framework regions LFR1, LFR2 and LFR3 of IGKV2-40*01 set forth in SEQ ID NO: 9, and a framework region LFR4 of IGKJ2*01 set forth in SEQ ID NO: 10, wherein the framework regions are determined according to a Kabat numbering scheme.

In some embodiments, the heavy chain framework region, numbered according to the Kabat numbering scheme, comprises a mutation of an amino acid residue at a position selected from positions 1, 44, 69, 71, 73, and 93, and preferably, the mutation includes: Q1E, R71A, and A93T; Q1E, I69L, R71A, and A93T; or Q1E, R44G, I69L, R71A, T73K, and A93T.

In some embodiments, the light chain framework region, numbered according to the Kabat numbering scheme, comprises at most one mutation of an amino acid residue; preferably, the mutation is a mutation of an amino acid residue at a position selected from position 2; and preferably, the mutation is I2V.

In some embodiments, the antibody or the antigen-binding fragment comprises: (1) a heavy chain variable region set forth in any one of SEQ ID NOs: 4-6, or a sequence having at least 80%, 85%, 90%, 91%, 92%, 93%, 94%, 95%, 96%, 97%, 98%, 99%, or 100% identity to the sequence set forth in any one of SEQ ID NOs: 4-6; or a sequence having at most 20, 19, 18, 17, 16, 15, 14, 13, 12, 11, 10, 9, 8, 7, 6, 5, 4, 3, 2, or 1 mutation compared with the sequence set forth in any one of SEQ ID NOs: 4-6; the mutation can be selected from an insertion, a deletion, and/or a substitution, and preferably, the substitution is a conservative amino acid substitution;

and/or, (2) a light chain variable region set forth in any one of SEQ ID NOs: 7-8 and 13-14, or a sequence having at least 80%, 85%, 90%, 91%, 92%, 93%, 94%, 95%, 96%, 97%, 98%, 99%, or 100% identity to the sequence set forth in any one of SEQ ID NOs: 7-8 and 13-14; or a sequence having at most 20, 19, 18, 17, 16, 15, 14, 13, 12, 11, 10, 9, 8, 7, 6, 5, 4, 3, 2, or 1 mutation compared with the sequence set forth in any one of SEQ ID NOs: 7-8 and 13-14; the mutation can be selected from an insertion, a deletion, and/or a substitution, and preferably, the substitution is a conservative amino acid substitution.

Preferably, the antibody or the antigen-binding fragment comprises: (1) a heavy chain variable region having a sequence set forth in SEQ ID NO: 4, 5, or 6, and a light chain variable region having a sequence set forth in SEQ ID NO: 14; or

(2) a heavy chain variable region having a sequence set forth in SEQ ID NO: 5, and a light chain variable region having a sequence set forth in SEQ ID NO: 7; or
(3) a heavy chain variable region and a light chain variable region having sequences having at least 70%, 75%, 80%, 85%, 90%, 95%, 96%, 97%, 98%, 99% or more identity to the sequences set forth in (1) and (2) above, respectively.

In some embodiments, the antibody or the antigen-binding fragment comprises a sequence having at least 80%, 85%, 90%, 91%, 92%, 93%, 94%, 95%, 96%, 97%, 98%, 99%, or 100% identity to the CDR1, the CDR2, and/or the CDR3; or a sequence having 1, 2, 3 or more amino acid insertions, deletions and/or substitutions compared with the CDR1, the CDR2, and/or the CDR3, and preferably, the substitutions are conservative amino acid substitutions.

In some embodiments, the antibody or the antigen-binding fragment specifically binds to a human and/or monkey GPC3 protein; and preferably, the antibody or the antigen-binding fragment binds to the human and/or monkey GPC3 with a dissociation constant (KD) not greater than 1.00E-7 M, 1.00E-8 M, 2.00E-8 M, 3.00E-8 M, 4.00E-8 M, 5.00E-8 M, 6.00E-8 M, 7.00E-8 M, 8.00E-8 M, 9.00E-8 M, 1.00E-9 M, 2.00E-9 M, 3.00E-9 M, 4.00E-9 M, 5.00E-9 M, 6.00E-9 M, 7.00E-9 M, 8.00E-9 M, 9.00E-9 M, or 6.00E-10 M.

In some embodiments, the antibody or the antigen-binding fragment is selected from full-length antibodies, VH single domain antibodies, Fab fragments, Fab′ fragments, F(ab)′2 fragments, Fd fragments, Fv fragments, complementarity determining region (CDR) fragments, single chain variable fragments (scFvs), scFv2, disulfide stabilized variable fragments (dsFvs), domain antibodies, bivalent single chain antibodies, single chain phage antibodies, bispecific diabodies, triabodies, tetrabodies, and minimal recognition units of antibodies.

According to another aspect, the present invention provides an immunoconjugate comprising any one of the foregoing antibodies or antigen-binding fragments and an effector molecule; and preferably, the effector molecule is linked to the antibody or the antigen-binding fragment.

In some embodiments, the effector molecule includes a therapeutic agent or a marker; and preferably, the therapeutic agent is selected from a drug, a toxin, a radioisotope, a chemotherapeutic drug, and an immunomodulator.

In some embodiments, the immunoconjugate further comprises a linker for conjugating the effector molecule to the antibody or the antigen-binding fragment, and the linker includes but is not limited to hydrazones, thioethers, esters, disulfides, and peptide-containing linkers.

According to another aspect, the present invention provides a chimeric antigen receptor (CAR) comprising an extracellular antigen-binding domain, a transmembrane domain, and an intracellular signaling domain, and the extracellular antigen-binding domain comprises the antibody or the antigen-binding fragment according to any one of the foregoing embodiments. According to another aspect, the present invention provides an immunocompetent cell, the immunocompetent cell expresses any one of the foregoing chimeric antigen receptors or comprises a nucleic acid molecule encoding any one of the foregoing chimeric antigen receptors; and preferably, the immunocompetent cell is selected from: a T cell, a natural killer cell (NK cell), a natural killer T cell (NKT cell), a double negative T cell (DNT cell), a monocyte, a macrophage, a dendritic cell, and a mast cell, and the T cell is preferably selected from a cytotoxic T cell, a regulatory T cell, and a helper T cell.

According to another aspect, the present invention provides a multispecific molecule comprising any one of the foregoing antibodies or antigen-binding fragments; and preferably, the multispecific molecule further comprises an antibody or an antigen-binding fragment specifically binding to an antigen other than GPC3 or binding to a GPC3 epitope different from any one of the foregoing antibodies or antigen-binding fragments.

In some embodiments, the antigen other than GPC3 is an antigen on surface of a T cell, a B cell, a natural killer cell, a dendritic cell, a macrophage, a monocyte, or a neutrophil; and preferably, the antigen other than GPC3 is selected from: CD3, CD3γ, CD3δ, CD3ε, CD3ζ, CD16, CD16A, CD32B, PD-1, PD-2, PD-L1, VEGF, NKG2D, CD19, CD20, CD40, CD47, 4-1BB, CD137, EGFR, EGFRvIII, TNF-alpha, CD33, HER2, HER3, HAS, CD5, CD27, EphA2, EpCAM, MUC1, MUC16, CEA, Claudin18.2, a folate receptor, Claudin6, WT1, NY-ESO-1, MAGE3, ASGPR1, and CDH16.

In some embodiments, the multispecific molecule is a tandem scFv, a bifunctional antibody (Db), a single chain bifunctional antibody (scDb), a dual affinity retargeting (DART) antibody, F(ab′)2, a dual variable domain (DVD) antibody, a knobs-into-holes (KiH) antibody, a dock-and-lock (DNL) antibody, a chemically cross-linked antibody, a heteropolyantibody or a heteroconjugate antibody.

According to another aspect, the present invention provides an isolated nucleic acid molecule encoding any one of the foregoing antibodies or antigen-binding fragments, any one of the foregoing chimeric antigen receptors, and any one of the foregoing multispecific molecules.

According to another aspect, the present invention provides a vector comprising the nucleic acid molecule.

According to another aspect, the present invention provides a host cell comprising the nucleic acid molecule or the expression vector, and preferably, the host cell is a prokaryotic cell or a eukaryotic cell, comprising a bacteria (E. coli), a fungus (yeast), an insect cell, or a mammalian cell (CHO cell line or 293T cell line).

According to another aspect, the present invention provides a method for preparing any one of the foregoing antibodies or antigen-binding fragments, or multispecific molecules, comprising: culturing the foregoing host cell, and isolating an antibody or an antigen-binding fragment expressed by the cell, or isolating a multispecific molecule expressed by the cell.

According to another aspect, the present invention provides a method for preparing the immunocompetent cell, comprising: introducing a nucleic acid fragment encoding any one of the foregoing chimeric antigen receptors into the immunocompetent cell, and optionally, the method further comprises initiating expression of any one of the foregoing chimeric antigen receptors by the immunocompetent cell.

According to another aspect, the present invention provides a pharmaceutical composition comprising a therapeutically effective amount of one or a combination of: any one of the foregoing antibodies or antigen-binding fragments; any one of the foregoing immunoconjugates; any one of the foregoing immunocompetent cells; any one of the foregoing multispecific molecules; any one of the foregoing nucleic acid molecules, expression vectors or host cells; or a product prepared by any one of the foregoing methods, and a pharmaceutically acceptable carrier.

According to another aspect, the present invention further discloses use of any one of the foregoing antibodies or antigen-binding fragments, the immunoconjugate, the immunocompetent cell, the multispecific molecule, the nucleic acid molecule, the expression vector, the product prepared by the method, or the pharmaceutical composition in preparation of a drug for treatment of a GPC3-mediated tumor; wherein the tumor is preferably selected from diseases such as hepatocellular carcinoma, melanoma, ovarian clear cell carcinoma, hepatoblastoma, neuroblastoma, Wilms' tumor, small cell lung cancer, lung adenocarcinoma, gastric cancer, colon cancer, rectal cancer, cervical cancer, breast cancer, ovarian cancer, skin cancer, lymphoma, prostate cancer, pancreatic cancer, renal cancer, esophageal cancer, thyroid cancer, testicular cancer, bladder cancer, bronchogenic carcinoma, nasopharyngeal cancer, head and neck cancer, endometrial cancer, brain cancer, bone cancer, leukemia, malignant mesothelioma, and liposarcoma; and preferably, hepatocellular carcinoma.

According to another aspect, the present invention further provides a method for treating a subject suffering from a GPC3-mediated tumor, comprising: selecting a subject suffering from a cancer expressing GPC3, and administering to the subject a therapeutically effective amount of any one of the foregoing antibodies or antigen-binding fragments, the immunoconjugate, the immunocompetent cell, the multispecific molecule, the nucleic acid molecule, the expression vector, the product prepared by the method, or the pharmaceutical composition; wherein the tumor is preferably selected from diseases such as hepatocellular carcinoma, melanoma, ovarian clear cell carcinoma, hepatoblastoma, neuroblastoma, Wilms' tumor, small cell lung cancer, lung adenocarcinoma, gastric cancer, colon cancer, rectal cancer, cervical cancer, breast cancer, ovarian cancer, skin cancer, lymphoma, prostate cancer, pancreatic cancer, renal cancer, esophageal cancer, thyroid cancer, testicular cancer, bladder cancer, bronchogenic carcinoma, nasopharyngeal cancer, head and neck cancer, endometrial cancer, brain cancer, bone cancer, leukemia, malignant mesothelioma, and liposarcoma; and preferably, hepatocellular carcinoma.

According to another aspect, the present invention further provides use of any one of the foregoing antibodies or antigen-binding fragments, the immunoconjugate, the immunocompetent cell, the multispecific molecule, the nucleic acid molecule, the expression vector, the product prepared by the method, or the pharmaceutical composition in treatment of a GPC3-positive tumor or cancer; wherein the tumor is preferably selected from diseases such as hepatocellular carcinoma, melanoma, ovarian clear cell carcinoma, hepatoblastoma, neuroblastoma, Wilms' tumor, small cell lung cancer, lung adenocarcinoma, gastric cancer, colon cancer, rectal cancer, cervical cancer, breast cancer, ovarian cancer, skin cancer, lymphoma, prostate cancer, pancreatic cancer, renal cancer, esophageal cancer, thyroid cancer, testicular cancer, bladder cancer, bronchogenic carcinoma, nasopharyngeal cancer, head and neck cancer, endometrial cancer, brain cancer, bone cancer, leukemia, malignant mesothelioma, and liposarcoma; and preferably, hepatocellular carcinoma.

According to another aspect, the present invention further provides a kit, comprising any one of the foregoing antibodies or antigen-binding fragments, any one of the foregoing immunoconjugates, the foregoing immunocompetent cell, any one of the foregoing multispecific molecules, the foregoing nucleic acid molecule, the foregoing expression vector, the product prepared by any one of the foregoing methods, or the foregoing pharmaceutical composition. According to another aspect, the present invention further provides use of any one of the foregoing antibodies or antigen-binding fragments in preparation of a reagent for detection or diagnosis of a tumor with a high expression of GPC3.

According to another aspect, the present invention further provides a method for detecting GPC3 expression in a biological sample, comprising: exposing a sample from a subject to any one of the foregoing antibodies or antigen-binding fragments, and detecting binding of the antibody or the antigen-binding fragment to the sample.

### Terminology and Definitions

Unless otherwise stated, the terms used herein have the meanings that are commonly understood by those of ordinary skill in the art. For a term explicitly defined herein, the meaning of the term shall be subject to the definition.

Furthermore, unless otherwise stated herein, terms used in the singular form herein shall include the plural form, and vice versa. More specifically, as used in this specification and the appended claims, unless otherwise clearly indicated, the singular forms “a”, “an”, and “the” include referents in the plural form.

As used herein, the terms “including”, “comprising” and “having” are used interchangeably and are intended to indicate the inclusion of a solution, implying that there may be elements other than those listed in the solution. Meanwhile, it should be understood that the descriptions “including”, “comprising”, and “having” as used herein also provide the solution of “consisting of . . . ”.

The term “and/or” as used herein includes the meanings of “and”, “or”, and “all or any other combination of elements linked by the term”.

The term “GPC3” or “glypican-3” herein is a member of the glypican family of heparan sulfate (HS) proteoglycans, which is attached to the cell surface by a glycosylphosphatidylinositol anchor. Human GPC3 has four known subtypes (subtypes 1-4) whose nucleic acid and amino acid sequences are known, including GenBank IDs: NM_001164617 and NP_001158089 (subtype 1); NM_004484 and NP_004475 (subtype 2); NM_001164618 and NP_001158090 (subtype 3); and NM_001164619 and NP_001158091 (subtype 4). In some embodiments disclosed herein, the antibodies disclosed herein may bind to one or more of the four GPC3 subtypes, or conservative variants thereof.

The term “specific binding” herein means that an antigen-binding molecule (e.g., an antibody) specifically binds to an antigen and substantially identical antigens, generally with high affinity, but does not bind to unrelated antigens with high affinity. Affinity is generally reflected in an equilibrium dissociation constant (KD), where a low KD indicates a high affinity. In the case of antibodies, high affinity generally means having a KD of about 1.00E-7M or less, about 1.00E-8M or less, about 1.00E-9M or less, or about 1.00E-10M or less. KD is calculated as follows: KD=Kd/Ka, where Kd represents the dissociation rate and Ka represents the association rate. The equilibrium dissociation constant KD can be measured by methods well known in the art, such as surface plasmon resonance (e.g., Biacore) or equilibrium dialysis.

The term “antigen-binding molecule” herein is used in the broadest sense and refers to a molecule that specifically binds to an antigen. For example, antigen-binding molecules include but are not limited to, antibodies or antibody mimetics. “Antibody mimetic” refers to an organic compound or a binding domain that is capable of specifically binding to an antigen, but is not structurally related to an antibody. For example, antibody mimetics include but are not limited to, affibody, affitin, affilin, a designed ankyrin repeat protein (DARPin), a nucleic acid aptamer, or a Kunitz domain peptide.

The term “antibody” herein is used in the broadest sense and refers to a polypeptide or a combination of polypeptides that comprises sufficient sequence from an immunoglobulin heavy chain variable region and/or sufficient sequence from an immunoglobulin light chain variable region to be capable of specifically binding to an antigen. “Antibody” herein encompasses various forms and various structures as long as they exhibit the desired antigen-binding activity. “Antibody” herein includes alternative protein scaffolds or artificial scaffolds having grafted complementarity determining regions (CDRs) or CDR derivatives. Such scaffolds include antibody-derived scaffolds comprising mutations introduced to, e.g., stabilize the three-dimensional structure of the antibody, and fully synthetic scaffolds comprising, e.g., biocompatible polymers. See, e.g., Korndorfer et al., 2003, Proteins: Structure, Function, and Bioinformatics, 53(1): 121-129 (2003); and Roque et al., Biotechnol. Prog. 20:639-654 (2004). Such scaffolds may also include non-antibody derived scaffolds, such as scaffold proteins known in the art to be useful for grafting CDRs, including but not limited to tenascin, fibronectin, peptide aptamers, and the like.

The term “antibody” herein includes a typical “tetrabody”, which belongs to an immunoglobulin consisting of two heavy chains (HCs) and two light chains (LCs). The heavy chain refers to a polypeptide chain consisting of, from the N-terminus to the C-terminus, a heavy chain variable region (VH), a heavy chain constant region CH1 domain, a hinge region (HR), a heavy chain constant region CH2 domain, and a heavy chain constant region CH3 domain; moreover, when the antibody is of IgE isotype, the heavy chain optionally further comprises a heavy chain constant region CH4 domain. The light chain is a polypeptide chain consisting of, from the N-terminus to the C-terminus, a light chain variable region (VL) and a light chain constant region (CL). The heavy chains are linked to each other and to the light chains through disulfide bonds to form a Y-shaped structure. The heavy chain constant regions of an immunoglobulin differ in their amino acid composition and arrangement, and thus in their antigenicity. Accordingly, “immunoglobulin” herein may be divided into five classes, or isotypes of immunoglobulins, i.e., IgM, IgD, IgG, IgA, and IgE, with their corresponding heavy chains being μ, δ, γ, α, and ε chains, respectively. The Ig of the same class can be divided into different subclasses according to the differences in the amino acid composition of the hinge regions and the number and location of disulfide bonds in the heavy chains. For example, IgG can be divided into IgG1, IgG2, IgG3, and IgG4; and IgA can be divided into IgA1 and IgA2. Light chains are divided into κ or λ chains according to differences in the constant regions. Each of the five classes of Ig may have a κ chain or λ chain.

The term “antibody” herein may be derived from any animal, including but not limited to human and non-human animals which may be selected from primates, mammals, rodents, and vertebrates, such as Camelidae species, Lama glama, Lama guanicoe, Vicugna pacos, sheep, rabbits, mice, rats, or Chondrichthyes (e.g., shark).

The term “antibody” herein includes but is not limited to, monoclonal antibodies, polyclonal antibodies, monospecific antibodies, multispecific antibodies (e.g., bispecific antibodies), monovalent antibodies, multivalent antibodies, intact antibodies, antigen-binding fragments, naked antibodies, conjugated antibodies, humanized antibodies, or fully human antibodies.

The term “antigen-binding fragment” herein refers to one or more antibody fragments that retain the ability to specifically bind to a target antigen. The antigen-binding function of an antibody may be performed by fragments of a full-length antibody. An antibody fragment may be an Fab, F(ab′)2, scFv, SMIP, diabody, triabody, affibody, nanobody, aptamer or domain antibody. Examples of binding fragments encompassing the term “antigen-binding fragment” of an antibody include, but are not limited to: (i) an Fab fragment, i.e., a monovalent fragment consisting of VL, VH, CL and CH1 domains; (ii) an F(ab)2 fragment, i.e., a bivalent fragment comprising two Fab fragments linked by a disulfide bond at the binge region; (iii) an Fd fragment consisting of VH and CH1 domains; (iv) an Fv fragment consisting of VL and VH domains of a single arm of an antibody; (V) a dAb comprising VH and VL domains; (vi) a dAb fragment consisting of a VH domain (Ward et al., Nature 341:544-546, 1989) or VHH; (vii) a dAb consisting of a VH or VL domain; (viii) an isolated complementarity determining region (CDR); (ix) a heavy chain antibody fragment consisting of VHH, CH2, and CH3; and (x) a combination of two or more isolated CDRs, which may optionally be joined by a synthetic linker. Furthermore, although the two domains (VL and VH) of the Fv fragment are encoded by separate genes, these two domains may be joined using a recombination method through a linker that enables them to be made into a single protein chain in which the VL and VH regions are paired to form a monovalent molecule (known as single chain Fv (scFv); see, e.g., Bird et al., Science 242:423-426, 1988 and Huston et al., Proc. Natl. Acad. Sci. USA 85:5879-5883, 1988). These antibody fragments may be obtained using a conventional technique known to those skilled in the art, and these fragments are screened for use in the same manner as intact antibodies. Antigen-binding fragments may be produced by a recombinant DNA technique or enzymatic or chemical cleavage of intact immunoglobulins or, in some embodiments, by a chemical peptide synthesis procedure known in the art.

The term “monoclonal antibody” herein refers to an antibody derived from a single clone (including any eukaryotic, prokaryotic, or phage clone), and is not limited to the production method of the antibody.

The term “multispecific” means having at least two antigen-binding sites, each of which binds to a different epitope of the same antigen or a different epitope of a different antigen. Thus, the terms such as “bispecific”, “trispecific”, and “tetraspecific” refer to the number of different epitopes to which an antibody/antigen-binding molecule can bind.

The term “valent” herein refers to the presence of a specified number of binding sites in an antibody/antigen-binding molecule. Thus, the terms “monovalent”, “divalent”, “tetravalent”, and “hexavalent” refer to the presence of one binding site, two binding sites, four binding sites, and six binding sites, respectively, in an antibody/antigen-binding molecule.

The term “scFv” (single-chain variable fragment) herein refers to a single polypeptide chain comprising VL and VH domains, wherein the VL and VH are linked through a linker (see, e.g., Bird et al., Science 242: 423-426 (1988); Huston et al., Proc. Natl. Acad. Sci. USA 85: 5879-5883 (1988); and Pluckthun, The Pharmacology of Monoclonal Antibodies, Vol. 113, Roseburg and Moore Ed., Springer-Verlag, New York, pp 269-315 (1994)). Such scFv molecules may have a general structure: NH2-VL-linker-VH—COOH or NH2-VH-linker-VL-COOH. An appropriate linker in the prior art consists of GGGGS amino acid sequence repeats or a variant thereof. For example, a linker having the amino acid sequence (GGGGS)4 can be used, and variants thereof can also be used (Holliger et al. (1993), Proc. Natl. Acad. Sci. USA 90: 6444-6448). Other linkers that can be used in the present invention are described in Alfthan et al. (1995), Protein Eng. 8: 725-731; Choi et al. (2001), Eur. J. Immunol. 31: 94-106; Hu et al. (1996), Cancer Res. 56: 3055-3061; Kipriyanov et al. (1999), J. Mol. Biol. 293: 41-56; and Roovers et al. (2001), Cancer Immunol. In some cases, there may also be disulfide bonds between the VH and VL of the scFv, forming a disulfide-linked Fv (dsFv).

The term “humanized antibody” herein refers to a genetically engineered non-human antibody that has an amino acid sequence modified to increase homology to the sequence of a human antibody. Generally, all or part of the CDR regions of a humanized antibody are derived from a non-human antibody (donor antibody), and all or part of the non-CDR regions (e.g., variable region FRs and/or constant regions) are derived from a humanized immunoglobulin (receptor antibody). The humanized antibody generally retains or partially retains the desired properties of the donor antibody, including but not limited to, antigen specificity, affinity, reactivity, the ability to increase the activity of immune cells, the ability to enhance an immune response, and the like.

The term “variable region” herein refers to a region of the heavy or light chain of an antibody involved in the binding of the antibody to an antigen. “Heavy chain variable region” is used interchangeably with “VH” and “HCVR”, and “light chain variable region” is used interchangeably with “VL” and “LCVR”. Heavy and light chain variable domains (VH and VL, respectively) of natural antibodies generally have similar structures, each of which contains four conserved framework regions (FRs) and three hypervariable regions (HVRs). See, e.g., Kindt et al., Kuby Immunology, 6th ed., W. H. Freeman and Co., p. 91 (2007). A single VH or VL domain may be sufficient to provide antigen-binding specificity. The terms “complementarity determining region” and “CDR” herein are used interchangeably and generally refer to a hypervariable region (HVR) of a heavy chain variable region (VH) or a light chain variable region (VL), which is also known as the complementarity determining region because it can form precise complementarity to an epitope in a spatial structure, wherein the heavy chain variable chain CDR may be abbreviated as HCDR and the light chain variable chain CDR may be abbreviated as LCDR. The terms “framework region” and “FR region” are used interchangeably to refer to those amino acid residues in a heavy chain variable region or a light chain variable region other than CDRs of an antibody, HFR may refer to the framework region of the heavy chain variable region, and LFR may refer to the framework region of the light chain variable region. Generally, a typical antibody variable region consists of 4 FR regions and 3 CDR regions in the following order: FR1-CDR1-FR2-CDR2-FR3-CDR3-FR4.

For further description of the CDRs, see Kabat et al., J. Biol. Chem., 252:6609-6616 (1977); Kabat et al., United States Department of Health and Human Services, “Sequences of proteins of immunological interest” (1991); Chothia et al., J. Mol. Biol. 196:901-917 (1987); Al-Lazikani B. et al., J. Mol. Biol., 273: 927-948 (1997); MacCallum et al., J. Mol. Biol. 262:732-745 (1996); Abhinandan and Martin, Mol. Immunol., 45: 3832-3839 (2008); Lefranc M. P. et al., Dev. Comp. Immunol., 27: 55-77 (2003); and Honegger and Plückthun, J. Mol. Biol., 309:657-670 (2001). “CDR” herein may be labeled and defined in a manner well known in the art, including but not limited to, Kabat numbering scheme, Chothia numbering scheme, or IMGT numbering scheme; the tool sites used include but are not limited to, AbRSA site (http://cao.labshare.cn/AbRSA/cdrs.php), abYsis site (www.abysis.org/abysis/sequence_input/key_annotation/key_annotation.cgi), and IMGT site (http://www.imgt.org/3Dstructure-DB/cgi/DomainGapAlign.cgi#results). The CDR herein includes overlaps and subsets of amino acid residues defined in different ways.

Unless otherwise specified, the numbering of amino acid residues of the “antibody” or “antigen-binding fragment” described herein is determined by the Kabat numbering scheme, as described in Kabat et al., Sequences of Proteins of Immunological Interest, 5th Ed. Public Health Service, National Institutes of Health, Bethesda, Md., 1991. The numbering of amino acid residues in the variable region of the antibody can be found at: http://www.abysis.org/abysis/sequence_input/key_annotation/key_annotation. cgi.

According to the Kabat numbering scheme
Framework regions LFR1, LFR2, LFR3, and
LFR4 of IGKV2-40*01 set forth in SEQ ID
NO: 9 and IGKJ2*01 set forth in
SEQ ID NO: 10:
LFR1 is
(SEQ ID NO: 22)
DIVMTQTPLSLPVTPGEPASISC;
LFR2 is
(SEQ ID NO: 23)
WYLQKPGQSPQLLIY;
LFR3 is
(SEQ ID NO: 24)
GVPDRFSGSGSGTDFTLKISRVEAEDVGVYYC;
and
LFR4 is
(SEQ ID NO: 10)
FGQGTKLEIK.
Framework regions HFRI, HFR2,
HFR3, and HFR4 of IGHV1-3*01
set forth in SEQ ID NO: 11 and
IGHJ1*01 set forth in SEQ ID NO: 12:
HFR1 is
(SEQ ID NO: 25)
QVQLVQSGAEVKKPGASVKVSCKASGYTFT;
HFR2 is
(SEQ ID NO: 26)
WVRQAPGQRLEWMG;
HFR3 is
(SEQ ID NO: 27)
RVTITRDTSASTAYMELSSLRSEDTAVYYCAR;
and
HFR4 is
(SEQ ID NO: 12)
WGQGTLVTVSS.

The term “conservative amino acid” herein generally refers to amino acids that belong to the same class or have similar characteristics (e.g., charge, side chain size, hydrophobicity, hydrophilicity, backbone conformation, and rigidity). For example, the amino acids in each of the following groups are conservative amino acid residues of each other, and substitutions of amino acid residues within the groups are conservative amino acid substitutions:
1) alanine (A), serine (S), and threonine (T);
2) aspartic acid (D) and glutamic acid (E);
3) asparagine (N) and glutamine (Q);
4) arginine (R), lysine (K), and histidine (H);
5) isoleucine (I), leucine (L), methionine (M), and valine (V); and
6) phenylalanine (F), tyrosine (Y), and tryptophan (W).

The terms “identity” and “sequence . . . identity” herein are interchangeable and are obtained by calculating as follows: to determine the percent “identity” of two amino acid sequences or two nucleic acid sequences, the sequences are aligned for optimal comparison purposes (e.g., for optimal alignment, gaps can be introduced in one or both of the first and second amino acid sequences or nucleic acid sequences, or non-homologous sequences can be discarded for comparison). Amino acid residues or nucleotides at corresponding amino acid positions or nucleotide positions are then compared. When a position in the first sequence is occupied by the same amino acid residue or nucleotide at the corresponding position in the second sequence, the molecules are identical at this position.

The term “immunoconjugate” herein refers to a polypeptide molecule containing at least one effector molecule and at least one antibody or a functional fragment thereof.

The term “effector molecule” herein is a part of an immunoconjugate, which is intended to have a desired effect on cells targeted by the immunoconjugate. An effector molecule is also referred to as an effector moiety (EM), a therapeutic agent, a diagnostic agent, a tracer, or a similar term.

The term “chimeric antigen receptor (CAR)” herein refers to an artificial cell surface receptor engineered to be expressed on an immunocompetent cell and specifically binds to an antigen, which comprises at least (1) an extracellular antigen-binding domain, e.g., a variable heavy chain or light chain of an antibody, (2) a transmembrane domain that anchors the CAR into the immunocompetent cell, and (3) an intracellular signaling domain. The CAR is capable of redirecting T cells and other immunocompetent cells to a selected target, e.g., a cancer cell, in a non-MHC-restricted manner using the extracellular antigen-binding domain.

The term “multispecific molecule” herein refers to a molecule having at least two antigen-binding sites, each of which binds to a different epitope of the same antigen or a different epitope of a different antigen. Thus, the terms such as “bispecific”, “trispecific”, and “tetraspecific” refer to the number of different epitopes to which an antibody/antigen-binding molecule can bind.

The term “immunocompetent cell” herein refers to a cell that performs immune functions in an organism. Examples of immunocompetent cells include: lymphocyte cells such as T cells, natural killer cells (NK cells), and B cells; antigen-presenting cells such as monocytes, macrophages, and dendritic cells; and granulocytes such as neutrophils, eosinophils, basophils, and mast cells. Specifically, T cells or NK cells from mammals such as human, dogs, cats, pigs, and mice are preferably enumerated, and preferably T cells or NK cells from human. In addition, T cells can be isolated and purified from body fluids such as blood and marrow fluids, from tissues such as spleen, thymus, and lymph nodes, or from immunocompetent cells infiltrating cancer tissues such as primary tumors, metastatic tumors, and malignant ascites. T cells made from ES cells and iPS cells can also be used. Examples of the T cells include α-β T cells, γ-δ T cells, CD8+ T cells, CD4+ T cells, tumor infiltrating T cells, memory T cells, naive T cells, and NKT cells. It should be noted that a source of immunocompetent cells and a subject to be administered may be the same or different. Then, when the subject to be administered is human, autologous cells derived from a patient as the subject to be administered, or allogeneic cells derived from another person may be used as the immunocompetent cells. That is, the donor and the receptor may or may not be identical, and preferably are identical.

The term “vector” herein refers to a nucleic acid molecule that is introduced into a host cell to produce a transformed host cell. The vector may contain a nucleic acid sequence (e.g., an origin of replication) that allows replication in the host cell. The vector may also contain one or more selectable marker genes and other genetic elements known in the art.

The term “host cell” herein refers to a cell in which a vector can proliferate and its DNA can be expressed, and the cell may be a prokaryotic cell or a eukaryotic cell. The term also includes any progeny of the subject host cell. It should be understood that not all progeny are identical to a parent cell, because mutations may occur during replication, such progeny are included.

The term “pharmaceutically acceptable carrier” herein includes any and all physiologically compatible solvents, dispersion media, coatings, antibacterial agents, antifungal agents, isotonic agents, absorption retardants, and analogs. Generally, the nature of the carrier depends on a specific method of administration used. For example, a parenteral formulation typically contains an injectable fluid as a vehicle, and the injectable fluid contains a pharmaceutically and physiologically acceptable fluid such as water, physiological saline, a balanced salt solution, aqueous dextrose, and glycerol. For solid compositions (e.g., in powder, pill, tablet, or capsule forms), conventional non-toxic solid carriers may include, for example, pharmaceutical mannitol, lactose, starch, or magnesium stearate. In addition to biologically neutral carriers, the pharmaceutical composition to be administered may also contain small amounts of non-toxic auxiliary substances such as a wetting agent or an emulsifier, a preservative, and a pH buffer, for example, sodium acetate or dehydrated sorbitan monolaurate.

The term “therapeutically effective amount” herein refers to an amount of the anti-GPC3 antibody or composition as disclosed herein that is effective in “treating” a disease or disorder of an individual. In the case of a cancer, a therapeutically effective amount of the anti-GPC3 antibody or composition as disclosed herein can reduce the number of cancer cells; reduce tumor size or weight; inhibit (i.e., slow down to some extent and preferably prevent) infiltration of cancer cells into surrounding organs; inhibit (i.e., slow down to some extent and preferably prevent) tumor metastasis; inhibit tumor growth to some extent; and/or alleviate, to some extent, one or more symptoms associated with the cancer. To the extent that the anti-GPC3 antibody or composition as disclosed herein can prevent growth and/or kill existing cancer cells, the anti-GPC3 antibody or composition may be cytostatic and/or cytotoxic. In some embodiments, the therapeutically effective amount is a growth inhibiting amount. In some embodiments, the therapeutically effective amount is an amount that prolongs survival of a patient. In some embodiments, the therapeutically effective amount is an amount that improves progression-free survival of a patient.

The ten “treatment” herein refers to a method for obtaining beneficial or desired outcomes (including clinical outcomes). For purposes of the present invention, beneficial or desired clinical outcomes include (but are not limited to) one or more of: alleviating one or more symptoms caused by the disease, attenuating the extent of the disease, stabilizing the disease (e.g., preventing or delaying disease progression), preventing or delaying disease spread (e.g., metastasis), preventing or delaying disease recurrence, delaying or slowing down disease progression, improving disease status, enabling disease remission (partial or complete), reducing the dose of one or more other drugs required to treat the disease, delaying disease progression, improving the quality of life, increasing weight gain, and/or prolonging survival. “Treatment” also encompasses a reduction in pathological outcomes (e.g., tumor volume) of a cancer. The method provided in the present invention encompasses any one or more of these therapeutic aspects.

The term “subject” herein refers to an organism that receives treatment for a particular disease or disorder described herein. Examples of subjects and patients include mammals, such as human, primates (e.g., monkey), or non-primate mammals, that receive treatment for a disease or disorder.

The term “diagnosis” herein refers to identification of the presence or nature of a pathological disorder, including but not limited to, liver cancer, ovarian cancer, melanoma, or lung cancer. Diagnostic methods vary in sensitivity and specificity. The “sensitivity” of a diagnostic assay is the percentage (percentage of true positives) of individual patients who are tested to be positive. The “specificity” of a diagnostic assay is 1 minus a false positive rate, where the false positive rate is defined as the proportion of those individuals who do not suffer from a disease but are tested to be positive. Although a particular diagnostic method may not provide a definitive diagnosis for a disorder, it is sufficient that the method can provide a positive indication to assist in diagnosis.

The term “hepatocellular carcinoma (HCC)” herein refers to a primary malignant liver tumor that typically occurs in patients with inflammatory livers caused by viral hepatitis, hepatotoxins, or cirrhosis (often caused by alcoholism). HCC is also referred to as malignant hepatoma.

### BRIEF DESCRIPTION OF THE DRAWINGS

FIG. 1A shows assay results for binding reactions of control antibodies with a human GPC3-His protein by ELISA;

FIG. 1B shows assay results for binding reactions of control antibodies with a monkey GPC3-His protein by ELISA;

FIG. 1C shows assay results for binding reactions of control antibodies with a murine GPC3-His protein by ELISA;

FIG. 2 shows assay results for binding reactions of control antibodies with a polypeptide GC3pep protein by ELISA;

FIG. 3 shows assay results for expression levels of GPC3 in HepG2 cells using antibody Y035 and antibody T2-23 by FACS;

FIG. 4 shows assay results for expression levels of GPC3 in CHO-K1-human GPC3 cells using antibody Y035 by FACS;

FIG. 5 shows assay results for expression levels of GPC3 in HEK293T-monkey GPC3 cells using antibody Y035 by FACS;

FIG. 6 shows assay results for binding reactions of anti-GPC3 humanized antibodies with a human GPC3-his protein by ELISA;

FIG. 7A shows assay results for binding reactions of anti-GPC3 humanized antibodies with CHO-K1-human GPC3 cells by FACS;

FIG. 7B shows assay results for binding reactions of anti-GPC3 humanized antibodies with CHO-K1 cells by FACS;

FIG. 8A shows assay results for binding reactions of anti-GPC3 humanized antibodies with HepG2 tumor cells by FACS;

FIG. 8B shows assay results for binding reactions of anti-GPC3 humanized antibodies with A431 tumor cells by FACS;

FIG. 9 shows assay results for binding reactions of anti-GPC3 humanized antibodies with a murine GPC3-his protein by ELISA;

FIG. 10 shows assay results for binding reactions of anti-GPC3 humanized antibodies with a monkey GPC3-His protein by ELISA;

FIG. 11A shows assay results for binding reactions of anti-GPC3 humanized antibodies with HEK293T-monkey GPC3 cells by FACS;

FIG. 11B shows assay results for binding reactions of anti-GPC3 humanized antibodies with HEK293T cells by FACS; and

FIG. 12 shows assay results for binding reactions of anti-GPC3 humanized antibodies with a GC3pep polypeptide protein by ELISA.

### DETAILED DESCRIPTION

The present invention will be further described with reference to specific examples, and the advantages and features of the present invention will become more apparent with the description. Experimental procedures without specified conditions in the examples are conducted according to conventional conditions or conditions recommended by the manufacturer. Reagents or instruments without specified manufacturers used herein are conventional products that are commercially available.

The examples are exemplary only and do not limit the scope of the present invention in any way. It will be understood by those skilled in the art that various changes or substitutions in form and details may be made to the technical solutions of the present invention without departing from the spirit and scope of the present invention, and that these changes and substitutions shall fall within the scope of the present invention.

The present invention will be further illustrated with reference to the following examples:

### Example 1: Preparation of Control Antibodies, Preparation of Human Polypeptides, Identification of Endogenous Cells, and Preparation of Over-Expressed Cell Strains

### (A) Preparation of Control Antibodies

Y035 and T2-23 clones are antibodies that recognize human GPC3 protein, both of which have strong binding affinity to the human GPC3 protein, and can also bind to cell lines with a high expression of GPC3, such as HepG2. Sequences of heavy chain variable regions and light chain variable regions of the Y035 and T2-23 clones are obtained according to the patent US2019/0046659A1. VLs and VHs of clones Y035 and T2-23 which recognize the human GPC3 were ligated to human IgG1 Fc in an order from an N-terminus to a C-terminus, respectively, wherein the VH and the VL were ligated by 3 GGGGS linkers to form scFv-human IgG1 Fc (scFv-hFc). The corresponding nucleotide sequences were cloned into a pTT5 vector (purchased from Youbio) respectively, and plasmids were prepared according to established standard molecular biology methods. See Sambrook, J., Fritsch, E. F., and Maniatis, T. (1989). Molecular Cloning: A Laboratory Manual, Second Edition (Plainview, New York: Cold Spring Harbor Laboratory Press). HEK293E cells (purchased from the Cell Bank of Type Culture Collection of Chinese Academy of Sciences) were transiently transfected with expression vectors according to PEI (purchased from Polysciences) instructions, cultured at 37° C. for 5 consecutive days using FreeStyle™ 293 (Invitrogen), and centrifuged to remove cell components to obtain culture supernatants containing antibodies in the form of ScFv-human IgG1 Fc (hFc). The culture supernatants were each loaded on a Protein A chromatography column (Protein A packing AT Protein A Diamond and column BXK16/26 were both purchased from Bestchrom), washed with a PBS phosphate buffer (pH 7.4), then washed with 20 mM PB and 1 M NaCl (pH 7.2), and finally eluted with a citrate buffer at pH 3.4. An Fc-tagged antibody eluted from the Protein A chromatography column was collected, neutralized with 1/10 volume of 1 M Tris at pH 8.0, and dialyzed with PBS at 4° C. overnight. The concentration of the dialyzed antibody was determined using Nanodrop, the purity of the antibody was determined using HPLC-SEC, the endotoxin content of the antibody was determined using an endotoxin assay kit (purchased from Zhanjiang A&C Biological Ltd.), and finally, the antibody was aseptically filtered through a 0.22 μM filter membrane, subpackaged, and stored at −80° C. Table 1 shows sequence information of the control antibodies, in which positions 1-113 of scFv-hFc of Y035 were a VL sequence, and positions 129-243 were a VH sequence; positions 1-111 of scFv-hFc of T2-23 were a VL sequence, and positions 127-247 were a VH sequence, as shown in underlined contents.

TABLE 1
Amino acid sequences of control antibodies

| Sequence | Sequence | |
|---|---|---|
| name | No. | Amino acid sequence |
| Y035 | NO: 1 | DIVMTQTPLSLPVITGEPAS |
| scFv-hFc | SEQ ID | ISCRSSQSLVHSNGNTYLQW |
| | | YLQKPGQSPQLLIYKVSNRF |
| | | SGVPDRESGSGSGTDFTLKI |
| | | SRVEAEDVGVYYCSQSIYVP |
| | | YTFGQGTKLEIKRGGGGGGG |
| | | GSGGGGSEVQLVQSGAEVKK |
| | | PGASVKVSCKASGYTFSDYE |
| | | MHWVRQAPGQGLEWMGAIHP |
| | | GSGDTAYNQRFKGRVTITAD |
| | | KSTSTAYMELSSLRSEDTAV |
| | | YYCARFYSYAYWGQGTLVTV |
| | | SAEPKSADKTHTCPPCPAPE |
| | | LLGGPSVFLFPPKPKDTLMI |
| | | SRTPEVTCVVVDVSHEDPEV |
| | | KFNWYVDGVEVHNAKTKPRE |
| | | EQYNSTYRVVSVLTVLHQDW |
| | | LNGKEYKCKVSNKALPAPIE |
| | | KTISKAKGQPREPQVYTLPP |
| | | SREEMTKNQVSLTCLVKGFY |
| | | PSDIAVEWESNGQPENNYKT |
| | | TPPVLDSDGSFFLYSKLTVD |
| | | KSRWQQGNVFSCSVMHEALH |
| | | NHYTQKSLSLSPGK |
| T2-23 | SEQ ID | QSALTQPPSASGSPGQSVTI |
| scFv-hFc | NO: 2 | SCTGTSSDVGGYNYVSWYQQ |
| | | YPGKAPKLLIYGNSNRPSGV |
| | | PDRESGSKSGTSASLAITGL |
| | | QAEDGADYYCQSYDSSLRVV |
| | | FGGGTKVTVLGGGGGSGGGG |
| | | SGGGGSQVQLQESGGGLVQP |
| | | GRSLRLSCAASGFTFSSYAM |
| | | HWVRQAPGKGLEWVSAISSS |
| | | GRSTYYADSVEGRFTISRDN |
| | | SKNTLYLQMNSLRAEDTAVY |
| | | YCAKDRRGSHADALNVWGQG |
| | | TLVTVSSEPKSADKTHTCPP |
| | | CPAPELLGGPSVFLFPPKPK |
| | | DTLMISRTPEVTCVVVDVSH |
| | | EDPEVKENWYVDGVEVHNAK |
| | | TKPREEQYNSTYRVVSVLTV |
| | | LHQDWLNGKEYKCKVSNKAL |
| | | PAPIEKTISKAKGQPREPQV |
| | | YTLPPSREEMTKNQVSLTCL |
| | | VKGFYPSDIAVEWESNGQPE |
| | | NNYKTTPPVLDSDGSFFLYS |
| | | KLTVDKSRWQQGNVFSCSVM |
| | | HEALHNHYTQKSLSLSPGK |

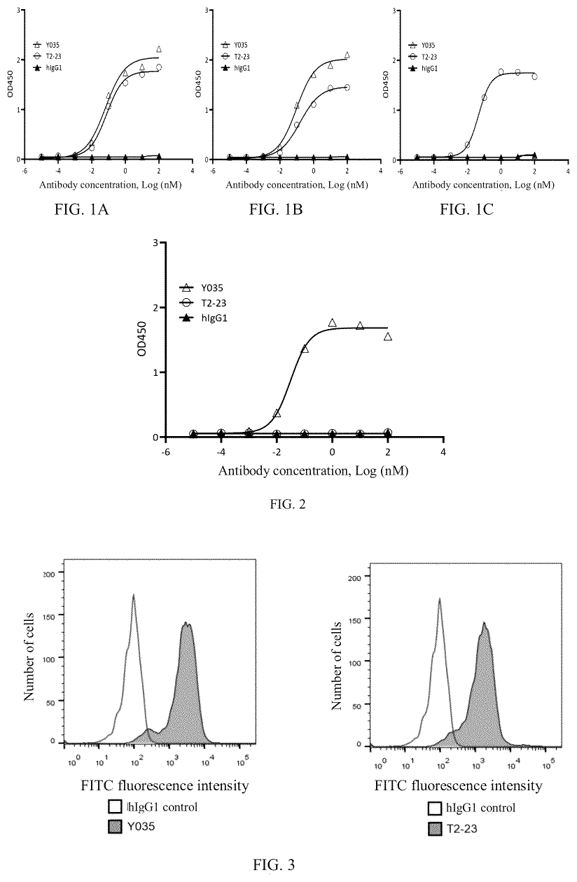

The binding activity of the control antibodies to a human GPC3-His protein (purchased from Acro, Cat. No. GP3-H52H4), a monkey GPC3-His protein (purchased from Acro, Cat. No. GP3-C5225), and a murine GPC3-His protein (purchased from Sino Biological, Cat. No. 50989-M08B) were assayed by ELISA. The specific method is described as follows: The antigen protein was diluted with PBS to a final concentration of 1 μg/mL, and the diluent was added to a 96-well ELISA plate at 50 μL/well. The plate was sealed with a plastic film and incubated at 4° C. overnight, washed twice with PBS the next day, and a blocking solution [PBS+2% (w/w) BSA] was added for blocking at room temperature for 2 h. The blocking solution was discarded, and 100 nM gradient-diluted control antibody or a negative control antibody was added at 50 μL/well. After incubation at 37° C. for 2 h, the plate was washed 3 times with PBS. A horseradish peroxidase (HRP)-labeled secondary antibody (purchased from Merck, Cat. No. AP113P) was added for incubation at 37° C. for 1 h, and the plate was washed 5 times with PBS. A TMB substrate was added at 50 μL/well for incubation at room temperature for 10 min, then a stop solution (1.0M HCl) was added at 50 μL/well. An ELISA plate reader (Multimode Plate Reader, EnSight, purchased from Perkin Elmer) was used to read OD₄₅₀ am values, with results as shown in Table 2, Table 3, Table 4, FIG. 1A, FIG. 1B, and FIG. 1C. The results showed that both the antibodies Y035 and T2-23 had a good binding activity with the human GPC3 protein and the monkey GPC3 protein; and the Y035 did not bind to the murine GPC3 protein, and the T2-23 bound well to the murine GPC3 protein. The IgG subtype control was human IgG1.

TABLE 2
Assay results for binding reactions of control
antibodies with human GPC3-his protein by ELISA

| OD450 | | | | | | | | |
|---|---|---|---|---|---|---|---|---|
| Antibody | Antibody concentration (nM) | | | | | | | |
| name | 100 | 10 | 1 | 0.1 | 0.01 | 0.001 | 0.0001 | blank |
| Y035 | 2.219 | 1.856 | 1.735 | 1.289 | 0.337 | 0.09 | 0.056 | 0.048 |
| T2-23 | 1.85 | 1.706 | 1.534 | 1.06 | 0.231 | 0.073 | 0.068 | 0.049 |
| hIgG1 | 0.072 | 0.05 | 0.048 | 0.05 | 0.048 | 0.048 | 0.047 | 0.049 |

TABLE 3
Assay results for binding reactions of control antibodies
with monkey GPC3-his protein by ELISA

| OD450 | | | | | | | | |
|---|---|---|---|---|---|---|---|---|
| Antibody | Antibody concentration (nM) | | | | | | | |
| name | 100 | 10 | 1 | 0.1 | 0.01 | 0.001 | 0.0001 | blank |
| Y035 | 2.103 | 1.888 | 1.706 | 1.091 | 0.254 | 0.072 | 0.047 | 0.046 |
| T2-23 | 1.446 | 1.431 | 1.103 | 0.694 | 0.131 | 0.057 | 0.044 | 0.046 |
| hIgG1 | 0.056 | 0.047 | 0.044 | 0.045 | 0.044 | 0.044 | 0.048 | 0.046 |

TABLE 4
Assay results for binding reactions of control antibodies
with murine GPC3-his protein by ELISA

| OD450 | | | | | | | | |
|---|---|---|---|---|---|---|---|---|
| Antibody | Antibody concentration (nM) | | | | | | | |
| name | 100 | 10 | 1 | 0.1 | 0.01 | 0.001 | 0.0001 | blank |
| Y035 | 0.09 | 0.064 | 0.049 | 0.056 | 0.054 | 0.05 | 0.058 | 0.051 |
| T2-23 | 1.672 | 1.761 | 1.77 | 1.252 | 0.303 | 0.086 | 0.05 | 0.052 |
| hIgG1 | 0.105 | 0.055 | 0.05 | 0.05 | 0.052 | 0.051 | 0.049 | 0.057 |

### (B) Preparation of Human Polypeptide GC3pep

Polypeptide GC3pep (AELAYDLDVDDAPGNSQQATPKDNEISTFHNLGNVHSPLK, SEQ ID NO: 3) of human GPC3 (NCBI: NM_004484.3, Ala524-Lys563) was produced. The prepared polypeptide was assayed by ELISA as described in Example 1 (A), respectively, with positive control antibodies which recognize different epitopes, with results as shown in Table 5 and FIG. 2. The results showed that the T2-23 could not bind to the polypeptide GC3pep, while the Y035 could bind to the polypeptide GC3pep, indicating that an antigenic polypeptide with binding activity with the antibodies has been prepared.

TABLE 5
Assay results for binding reactions of control antibodies
with polypeptide GC3pep protein by ELISA

| OD450 | | | | | | | | |
|---|---|---|---|---|---|---|---|---|
| Antibody | Antibody concentration (nM) | | | | | | | |
| name | 100 | 10 | 1 | 0.1 | 0.01 | 0.001 | 0.0001 | blank |
| Y035 | 1.554 | 1.722 | 1.769 | 1.366 | 0.373 | 0.088 | 0.056 | 0.049 |
| T2-23 | 0.069 | 0.049 | 0.059 | 0.051 | 0.046 | 0.065 | 0.063 | 0.048 |
| hIgG1 | 0.069 | 0.05 | 0.05 | 0.047 | 0.046 | 0.046 | 0.06 | 0.051 |

### (C) Identification of Cell Strains Endogenously Expressing GPC3 Protein

HepG2 cells were expanded to a logarithmic growth phase in a T-75 cell culture flask, the culture supernatant was discarded by centrifugation, and the cell pellet was washed twice with PBS. Assay and analysis were performed by FACS (FACS Canto™, purchased from BD Biosciences) using the antibodies Y035 and T2-23 as primary antibodies and an FITC-labeled secondary antibody (purchased from Invitrogen, Cat. No. A11013), with results as shown in Table 6 and FIG. 3. The results showed that HepG2 cells could bind to both the Y035 and the T2-23.

TABLE 6
Assay results for endogenous cell line HepG2 cells by FACS

| | | Mean fluorescence intensity of cells | |
|---|---|---|---|
| No. | Antibody name | IgG subtype control | GPC3 antibody |
| 1 | Y035 | 137 | 3647 |
| 2 | T2-23 | 137 | 2573 |

### (D) Preparation of CHO-K1 Recombinant Cell Strain Expressing Human GPC3 Protein

A nucleotide sequence encoding a full-length amino acid sequence (NCBI: NM_004484.3) of human GPC3 was cloned into a pcDNA3.1 vector (purchased from Clontech), and a plasmid was prepared. After plasmid transfection (Lipofectamine® 3000 Transfection Kit, purchased from Invitrogen, Cat. No. L3000-015), CHO-K1 cell lines (purchased from the Cell Bank of Type Culture Collection of Chinese Academy of Sciences) were subjected to selective culture in a DMEM/F12 medium containing 10% (w/w) fetal bovine serum and containing 10 μg/mL puromycin for 2 weeks, then positive monoclonal cells were sorted onto a 96-well plate on a flow cytometer (FACSAriaII, purchased from BD Biosciences) using an antibody Y035 and a goat anti-human IgG H+L antibody (Jackson, Cat. No. 109605088), and cultured in 5% (v/v) CO₂ at 37° C., and a portion of the monoclonal wells were selected for amplification approximately 2 weeks later. The amplified clones were screened by flow cytometry. Monoclonal cell lines with better growth and higher fluorescence intensity were selected for further expansion and cryopreserved in liquid nitrogen. The results of selection are shown in Table 7 and FIG. 4, in which the IgG subtype control was a human IgG1 control. Table 7 shows that a series of CHO-K1 monoclonal cell lines with positive expression of human GPC3 have been prepared. In FIG. 4, the abscissa represents the fluorescence intensity of the cells, and the ordinate represents the number of the cells. The results indicated that 1C3, 2B5, and 3E9 were cell strains with high-level expression of human GPC3. The cell strain, used in the present invention, with a high-level expression of human GPC3 was 2B5.

TABLE 7
Assay results for CHO-K1 recombinant cell lines
expressing human GPC3 protein by FACS

| | Clone No. of stably | Mean fluorescence intensity of cells | |
|---|---|---|---|
| No. | transfected cell line | IgG subtype control | GPC3 antibody |
| 1 | CHOK1-hGPC3.1C3 | 19.3 | 14300 |
| 2 | CHOK1-hGPC3.2B5 | 19.3 | 18900 |
| 3 | CHOK1-hGPC3.3E9 | 19.3 | 26700 |

### (E) Preparation of Recombinant HEK293T Cell Strains Expressing Monkey GPC3 Protein

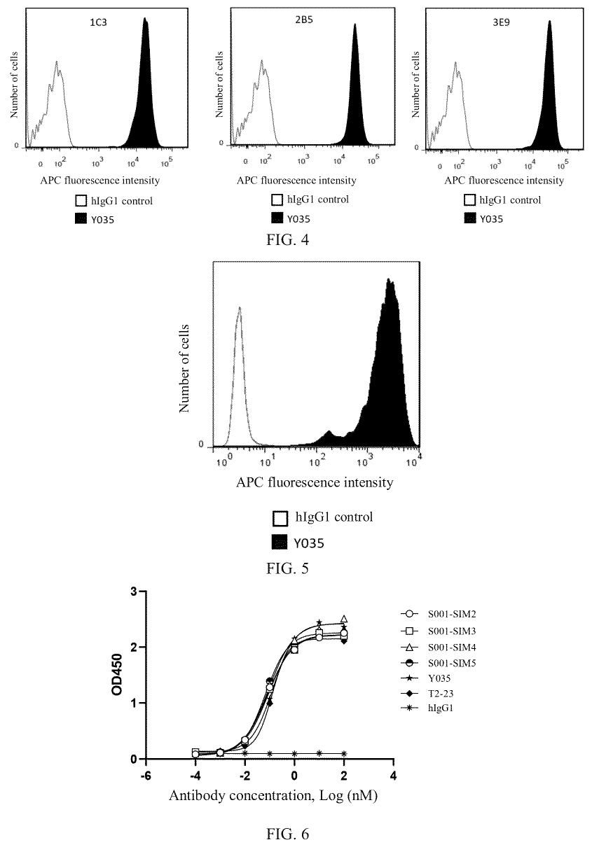

A nucleotide sequence encoding a full-length amino acid sequence (NCBI: XP_011739317.1) of monkey GPC3 was cloned into a pcDNA3.1 vector (purchased from Thermofisher scientific), and a plasmid was prepared. After plasmid transfection with FuGENE® HD (Promega, Cat. No. #E2311), HEK293T cell lines were subjected to selective culture in a DMEM medium containing 10% (w/w) fetal bovine serum and containing 10 μg/mL puromycin for 2 weeks, then enriched positive monoclonal cells were sorted onto a 96-well plate on a flow cytometer (FACSAriaII, purchased from BD Biosciences) using an antibody Y035 and a goat anti-human IgG H+L antibody (Jackson, Cat. No. 109605088), cultured in 5% (v/v) CO₂ at 37° C., and amplified about one week later. The amplified cells were assayed by flow cytometry, and cell strains with better growth and higher fluorescence intensity were selected for further expansion and cryopreserved in liquid nitrogen. The results of expression levels are shown in FIG. 5, indicating that HEK293T-monkey-GPC3 subjected to puromycin pressure screening had a single positive peak and could be used for assaying cross-activity of antibodies.

### Example 2: Humanization of Murine GPC3 Antibody

By comparing the IMGT database (http://imgt.cines.fr) for germline genes from heavy chain and light chain variable regions of human antibodies, germline genes, with high homology with the murine antibody, from heavy chain and light chain variable regions were respectively selected as templates, and CDRs of the murine antibody were respectively grafted into corresponding humanized templates to form a variable region sequence of FR1-CDR1-FR2-CDR2-FR3-CDR3-FR4. A humanized anti-GPC3 monoclonal antibody was obtained by back-mutating key amino acids in a framework sequence to amino acids corresponding to the murine antibody as needed to ensure the original affinity. The amino acid residues of CDRs of antibodies are usually identified and annotated by the Kabat numbering scheme.

With IGKV2-40*01 and IGKJ2*01 as humanized light chain templates, and IGHV1-3*01 and IGHJ1*01 as humanized heavy chain templates, CDRs of the murine GPC3 antibody were respectively grafted into the humanized templates to obtain corresponding humanized versions. Key amino acids in the FR region sequence of the anti-GPC3 humanized antibody were back-mutated to amino acids corresponding to the murine antibody as needed to ensure the original affinity. Detailed back mutation design is shown in Table 8.

TABLE 8
Back mutation design for GPC3 humanized antibodies

| VL | | VH | |
|---|---|---|---|
| GPC3.VL1 | Grafted | GPC3.VH1 | Q1E, R71A, A93T |
| GPC3.VL2 | I2V | GPC3.VH2 | Q1E, I69L, R71A, A93T |
| — | — | GPC3.VHS | Q1E, R44G, I69L, R71A, |
| | | | T73K, A93T |

Note:
Grafted denotes that the CDRs of the murine antibody are grafted into the human germline FR region sequences; and I2V denotes that I at position 2 of Grafted is back-mutated to V, and so on. The numbering of the back-mutated amino acids is determined by the Kabat numbering scheme.

Specific sequences of the variable regions of the anti-GPC3 humanized antibodies are as follows:

GPC3. VH1 has an amino acid sequence set forth
in SEQ ID NO: 4:
EVQLVQSGAEVKKPGASVKVSCKASGYTFTDYEMHWVRQAPGQRL
EWMGALDPKTGDTAYSQKFKGRVTITADTSASTAYMELSSLRSED
TAVYYCTRFYSYTYWGQGTLVTVSS
GPC3. VH2 has an amino acid sequence set forth
in SEQ ID NO: 5:
EVQLVQSGAEVKKPGASVKVSCKASGYTFTDYEMHWVRQAPGQRL
EWMGALDPKTGDTAYSQKFKGRVTLTADTSASTAYMELSSLRSED
TAVYYCTRFYSYTYWGQGTLVTVSS
GPC3. VH5 has an amino acid sequence set forth
in SEQ ID NO: 6:
EVQLVQSGAEVKKPGASVKVSCKASGYTFTDYEMHWVRQAPGQGL
EWMGALDPKTGDTAYSQKFKGRVTLTADKSASTAYMELSSLRSED
TAVYYCTRFYSYTYWGQGTLVTVSS
GPC3. VL1 has an amino acid sequence set forth
in SEQ ID NO: 7:
DIVMTQTPLSLPVTPGEPASISCRSSQSLVHSNGNTYLHWYLQKP
GQSPQLLIYKVSNRFSGVPDRFSGSGSGTDFTLKISRVEAEDVGV
YYCSQNTHVPPTFGQGTKLEIK
GPC3.VL2 has an amino acid sequence set forth
in SEQ ID NO: 8:
DVVMTQTPLSLPVTPGEPASISCRSSQSLVHSNGNTYLHWYLQKP
GQSPQLLIYKVSNRFSGVPDRFSGSGSGTDFTLKISRVEAEDVGV
YYCSQNTHVPPTFGQGTKLEIK
The humanized light chain template IGKV2-40*01
has an amino acid
sequence set forth in SEQ ID NO: 9:
DIVMTQTPLSLPVTPGEPASISCRSSQSLLDSDDGNTYLDWYLQK
PGQSPQLLIYTLSYRASGVPDRFSGSGSGTDFTLKISRVEAEDVG
VYYCMQRIEFP
The humanized light chain template IGKJ2*01
has an amino acid sequence
set forth in SEQ ID NO: 10:
FGQGTKLEIK
The humanized heavy chain template IGHV1-3*01
has an amino acid
sequence set forth in SEQ ID NO: 11:
QVQLVQSGAEVKKPGASVKVSCKASGYTFTSYAMHWVRQAPGQRL
EWMGWINAGNGNTKYSQKFQGRVTITRDTSASTAYMELSSLRSED
TAVYYCAR
The humanized heavy chain template IGHJ1*01
has an amino acid sequence
set forth in SEQ ID NO: 12:
WGQGTLVTVSS

An anti-GPC3 antibody has a site NG that is susceptible to chemical modification, and point mutations were performed on the NG to eliminate modification risks. In two of the examples, NGs of GPC3.VL1 and GPC3.VL2 were mutated respectively, and mutated sequences were named GPC3. VL1a and GPC3.VL2a, respectively.

GPC3. VLla has an amino acid
sequence set forth in SEQ ID NO: 13:
DIVMTQTPLSLPVTPGEPASISCRSSQSLVHSNANTYLHWYLQKP
GQSPQLLIYKVSNRFSGVPDRFSGSGSGTDFTLKISRVEAEDVGV
YYCSQNTHVPPTFGQGTKLEIK
GPC3. VL2a has an amino acid
sequence set forth in SEQ ID NO: 14:
DVVMTQTPLSLPVTPGEPASISCRSSQSLVHSNANTYLHWYLQKP
GQSPQLLIYKVSNRFSGVPDRFSGSGSGTDFTLKISRVEAEDVGV
YYCSQNTHVPPTFGQGTKLEIK

From the back mutation design for the light chain and heavy chain variable regions of the anti-GPC3 humanized antibodies, different light chain and heavy chain sequences were selected respectively for cross combination to finally obtain 4 anti-GPC3 humanized antibodies. Specific combinations are shown in Table 9. The form of anti-GPC3 humanized antibody scFv-human IgG1 Fc (scFv-hFc) was constructed according to the method described in Example 1, and the Fc region sequence is shown as a sequence set forth in SEQ ID NO: 1 at positions 244-475.

TABLE 9
Combinations of anti-GPC3 humanized antibodies

| Antibody name | VL | VH |
|---|---|---|
| S001-SIM2 | GPC3.VL1 | GPC3.VH2 |
| S001-SIM3 | GPC3.VL2a | GPC3.VH1 |
| S001-SIM4 | GPC3.VL2a | GPC3.VH2 |
| S001-SIM5 | GPC3.VL2a | GPC3.VH5 |

Analysis results for the VH and VL sequences of the 4 humanized antibodies according to the Kabat numbering scheme are shown in Table 10.

TABLE 10
Kabat analysis results for VII and
VL sequences of anti-GPC3
humanized antibodies

| Heavy chain/light | | | |
|---|---|---|---|
| chain variable | | | |
| regions | CDR1 | CDR2 | CDR3 |
| VH1/2/5 | DYEMH | ALDPKT | FYSYTY |
| (SEQ ID | SEQ ID | GDTAYS | SEQ ID |
| NO: | NO: 15 | QKFKG | NO: 17 |
| 4/5/6) | | SEQ ID | |
| | | NO: 16 | |
| VL1 | RSSQSL | KVSNRFS | SQNTHV |
| (SEQ ID | VHSNGN | SEQ ID | PPT |
| NO: 7) | TYLH | NO: 19 | SEQ ID |
| | SEQ ID | | NO: 20 |
| | NO: 18 | | |
| VL2a | RSSQSL | KVSNRFS | SQNTH |
| (SEQ ID | VHSNAN | SEQ ID | VPPT |
| NO: 14) | TYLH | NO: 19 | SEQ ID |
| | SEQ ID | | NO: 20 |
| | NO: 21 | | |

### Example 3: Identification of Anti-GPC3 Humanized Antibodies

### (A) Assay for Binding of Anti-GPC3 Humanized Antibodies to Human GPC3 Protein by Enzyme-Linked Immunosorbent Assay (ELISA)

ELISA and data analysis were performed according to the method described in Example 1 (A). An ELISA plate reader (Multimode Plate Reader, EnSight, purchased from Perkin Elmer) was used to read OD_{450 nm} values, with results for binding activity of the anti-GPC3 humanized antibodies with the human GPC3 protein as shown in FIG. 6 and Table 11. The results showed that the 4 antibodies bound well to the human GPC3 protein, of which the IgG control was hIgG1, and data in the table are OD_{450 nm} values.

TABLE 11
Assay results for binding reactions of anti-GPC3 humanized
antibodies with human GPC3 protein by ELISA

| | Antibody concentration (nM) | | | | | | | |
|---|---|---|---|---|---|---|---|---|
| | | | | | | | | Blank |
| Antibody | | | | | | | | con- |
| name | 100 | 10 | 1 | 0.1 | 0.01 | 0.001 | 0.0001 | trol |
| S001-SIM2 | 2.25 | 2.17 | 1.95 | 1.28 | 0.34 | 0.11 | 0.08 | 0.08 |
| S001-SIM3 | 2.2 | 2.25 | 1.95 | 1.25 | 0.32 | 0.11 | 0.12 | 0.12 |
| S001-SIM4 | 2.51 | 2.31 | 2.12 | 1.33 | 0.34 | 0.11 | 0.1 | 0.08 |
| S001-SIM5 | 2.26 | 2.27 | 2.04 | 1.39 | 0.34 | 0.12 | 0.49 | 0.09 |
| Y035 | 2.36 | 2.44 | 2.15 | 1.07 | 0.32 | 0.13 | 0.1 | 0.1 |
| T2-23 | 2.11 | 2.17 | 2.05 | 0.99 | 0.23 | 0.14 | 0.11 | 0.1 |
| hIgG1 | 0.09 | 0.1 | 0.09 | 0.09 | 0.1 | 0.09 | 0.09 | 0.08 |

### (B) Assay for Binding of Anti-GPC3 Humanized Antibodies to Cells Expressing GPC3 Protein and Cells not Expressing GPC3 Protein (Negative Cells) by Flow Cytometry (FACS)

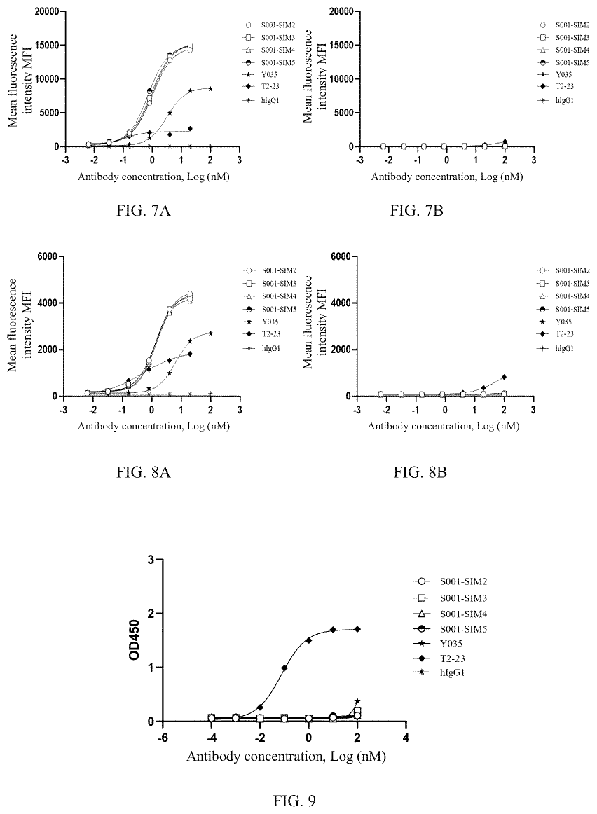

The desired cells were expanded to a logarithmic growth phase in a T-75 cell culture flask, the medium was removed by pipetting, and the cells were washed twice with a PBS buffer, and digested with trypsin. Then a complete medium was added to stop the digestion, and the cells were blown into a single cell suspension. After counting, the cells were centrifuged, and the cell pellet was resuspended with an FACS buffer (PBS+2% fetal bovine serum) to 2×10⁶ cells/mL. The cell resuspension was added to a 96-well FACS plate at 50 μL/well, and an anti-GPC3 humanized antibody sample to be tested was added at 50 μL/well for incubation at 4° C. for 1 h. After the plate was centrifuged and washed 3 times with a PBS buffer, a goat anti-human IgG H+L antibody (Jackson, Cat. No. 109605088) was added at 50 μL/well for incubation on ice for 1 h. After the plate was centrifuged and washed 3 times with a PBS buffer, and cells were resuspended in 100 μL of PBS, assay and analysis were performed by FACS (FACS Canto™, purchased from BD Biosciences). Data analysis was performed by software (FlowJo) to obtain the mean fluorescence intensity (MFI) of the cells. Then, analysis was performed by software (GraphPad Prism8), data were fitted, and EC₅₀ values were calculated. Table 12, FIG. 7A and FIG. 7B show that the 4 anti-GPC3 humanized antibodies could bind to CHO-K1-human GPC3 cells, but did not bind to CHOK1 cells; and Table 13, FIG. 8A and FIG. 8B show that the 4 anti-GPC3 humanized antibodies could bind to HepG2 cells, but did not bind to A431 cells.

TABLE 12
Assay results for binding reactions of anti-GPC3
humanized antibodies with CHO-K1-human
GPC3 cells and CHOK1 cells by FACS

| | CHOK1-hGPC3 | | CHOK1 | |
|---|---|---|---|---|
| | Maximum mean | | Maximum mean | |
| | fluorescence | | fluorescence | |
| Antibody | intensity | Ec50 | intensity | Ec50 |
| name | Max MFI | (nM) | Max MFI | (nM) |
| S001-SIM2 | 14285 | 1.00 | 92 | NB |
| S001-SIM3 | 14941 | 0.91 | 104 | NB |
| S001-SIM4 | 14895 | 0.99 | 94 | NB |
| S001-SIM5 | 14844 | 0.72 | 80 | NB |
| Y035 | 8548 | 3.51 | 74 | NB |
| T2-23 | 2680 | 0.11 | 766 | NB |
| hIgG1 | 82 | NB | 81 | NB |

TABLE 13
Assay results for binding reactions of anti-GPC3 humanized
antibodies with HepG2 cells and A431 cells by FACS

| | HepG2 | | A431 | |
|---|---|---|---|---|
| | Maximum mean | | Maximum mean | |
| | fluorescence | | fluorescence | |
| Antibody | intensity | Ec50 | intensity | Ec50 |
| name | Max MFI | (nM) | Max MFI | (nM) |
| S001-SIM2 | 4399 | 1.39 | 88 | NB |
| S001-SIM3 | 4215 | 1.31 | 107 | NB |
| S001-SIM4 | 4117 | 1.36 | 129 | NB |
| S001-SIM5 | 4295 | 1.38 | 88 | NB |
| Y035 | 2693 | 6.36 | 73 | NB |
| T2-23 | 1816 | 0.26 | 821 | NB |
| hIgG1 | 110 | NB | 90 | NB |

### Example 4: Assay for Cross-Binding Activity of Anti-GPC3 Humanized Antibodies

### (A) Assay for Binding of Anti-GPC3 Humanized Antibodies to Monkey GPC3 Protein and Murine GPC3 Protein by ELISA

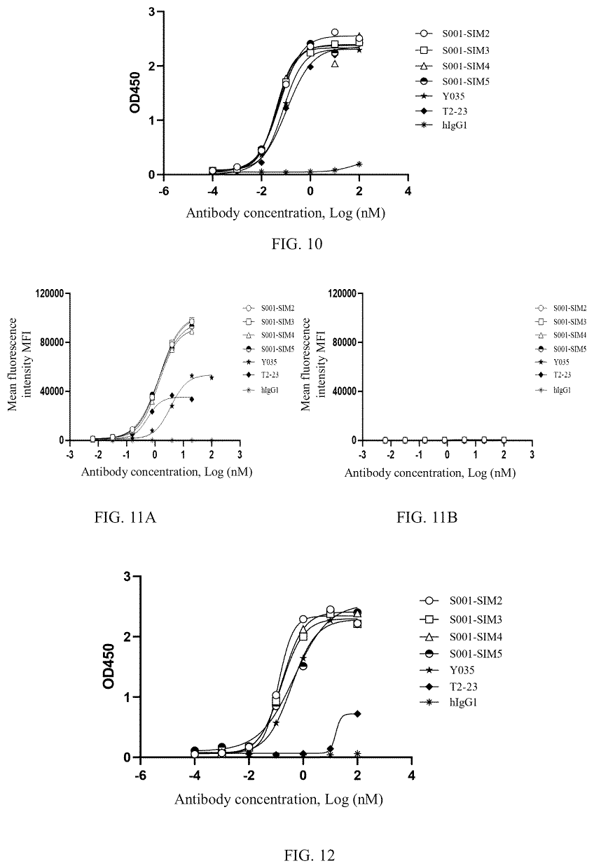

A monkey GPC3-His protein (purchased from Acro, Cat. No. GP3-C5225) and a murine GPC3-his protein (purchased from Sino Biological, Cat. No. 50989-M08B) were subjected to ELISA and data analysis according to the method described in Example 1 (A). The ELISA results for the anti-GPC3 humanized antibodies and the murine GPC3 protein are shown in FIG. 9 and Table 14. The results showed that the 4 anti-GPC3 humanized antibodies did not bind to the murine GPC3 protein. The IgG control was hIgG1, and data in the table are OD₄₅₀ values.

TABLE 14
Assay results for binding reactions of anti-GPC3 humanized
antibodies with murine GPC3 protein by ELISA

| | Antibody concentration (nM) | | | | | | | |
|---|---|---|---|---|---|---|---|---|
| | | | | | | | | Blank |
| Antibody | | | | | | | | con- |
| name | 100 | 10 | 1 | 0.1 | 0.01 | 0.001 | 0.0001 | trol |
| S001-SIM2 | 0.11 | 0.07 | 0.06 | 0.05 | 0.35 | 0.06 | 0.06 | 0.05 |
| S001-SIM3 | 0.2 | 0.05 | 0.06 | 0.07 | 0.06 | 0.07 | 0.07 | 0.04 |
| S001-SIM4 | 0.11 | 0.05 | 0.05 | 0.05 | 0.05 | 0.06 | 0.08 | 0.05 |
| S001-SIM5 | 0.1 | 0.1 | 0.05 | 0.66 | 0.07 | 0.08 | 0.07 | 0.08 |
| Y035 | 0.38 | 0.05 | 0.05 | 0.04 | 0.04 | 0.05 | 0.04 | 0.05 |
| T2-23 | 1.71 | 1.7 | 1.5 | 0.99 | 0.26 | 0.07 | 0.05 | 0.05 |
| hIgG1 | 0.08 | 0.07 | 0.05 | 0.05 | 0.05 | 0.05 | 0.05 | 0.05 |

The ELISA results for the anti-GPC3 humanized antibodies and the monkey GPC3 protein are shown in FIG. 10 and Table 15. The results showed that the 4 anti-GPC3 humanized antibodies bound well to the monkey GPC3 protein.

TABLE 15
Assay results for binding reactions of anti-GPC3 humanized
antibodies with monkey GPC3 protein by ELISA

| | Antibody concentration (nM) | | | | | | | |
|---|---|---|---|---|---|---|---|---|
| | | | | | | | | Blank |
| Antibody | | | | | | | | con- |
| name | 100 | 10 | 1 | 0.1 | 0.01 | 0.001 | 0.0001 | trol |
| S001-SIM2 | 2.51 | 2.62 | 2.36 | 1.66 | 0.44 | 0.14 | 0.07 | 0.06 |
| S001-SIM3 | 2.43 | 2.4 | 2.24 | 1.7 | 0.44 | 0.1 | 0.07 | 0.12 |
| S001-SIM4 | 2.55 | 2.04 | 2.37 | 1.77 | 0.44 | 0.1 | 0.07 | 0.05 |
| S001-SIM5 | 2.42 | 2.24 | 2.41 | 1.71 | 0.47 | 0.11 | 0.07 | 0.09 |
| Y035 | 2.29 | 2.25 | 2.25 | 1.31 | 0.36 | 0.1 | 0.06 | 0.05 |
| T2-23 | 2.45 | 2.2 | 1.98 | 1.22 | 0.22 | 0.07 | 0.05 | 0.05 |
| hIgG1 | 0.19 | 0.08 | 0.05 | 0.04 | 0.05 | 0.04 | 0.05 | 0.05 |

### (B) Assay for Binding of Anti-GPC3 Humanized Antibodies to Cells Expressing Monkey GPC3 Protein by ELISA

HEK293T-monkey GPC3 cells were subjected to FACS assay and data analysis according to the method described in Example 3 (B). The analysis results are shown in Table 16, FIG. 11A and FIG. 11B. The results showed that the 4 anti-GPC3 humanized antibodies bound to the HEK293T monkey-GPC3 cells, but did not bind to the HEK293T cells.

TABLE 16
Assay results for binding reactions of anti-GPC3
humanized antibodies with HEK293T-monkey GPC3
cells and HEK293T cells by FACS

| | HEK293T-cynoGPC3 | | HEK293T | |
|---|---|---|---|---|
| | Maximum mean | | Maximum mean | |
| | fluorescence | | fluorescence | |
| Antibody | intensity | Ec50 | intensity | Ec50 |
| name | Max MFI | (nM) | Max MFI | (nM) |
| S001-SIM2 | 97088 | 1.43 | 558 | NB |
| S001-SIM3 | 98105 | 1.42 | 584 | NB |
| S001-SIM4 | 89200 | 1.35 | 565 | NB |
| S001-SIM5 | 93197 | 1.30 | 598 | NB |
| Y035 | 52715 | 5.24 | 388 | NB |
| T2-23 | 36715 | 0.56 | 490 | NB |
| hIgG1 | 95 | NB | 57 | NB |

### Example 5: Assay for Affinity of Anti-GPC3 Humanized Antibodies

### (A) Assay for Affinity of Anti-GPC3 Humanized Antibodies to Human GPC3 Protein

Anti-GPC3 humanized antibodies were captured using a Protein A chip (GE Healthcare; 29-127-558). The sample and running buffer was HBS-EP+ (10 mM HEPES, 150 mM NaCl, 3 mM EDTA, 0.05% surfactant P20) (GE Healthcare; BR-1006-69). The flow cell was set at 25° C. The sample block was set at 16° C. Both were pretreated with the running buffer. In each cycle, first, the antibody to be tested was captured using the Protein A chip, and then a single concentration of GPC3 antigen protein was injected. The association and dissociation processes of the antibody with the antigen protein were recorded, and finally, the chip was regenerated using Glycine pH 1.5 (GE Healthcare; BR-1003-54). The association was determined by injecting different concentrations of human GPC3-His in the solution and maintaining for 240 s, wherein the flow rate was 30 μL/min, and the protein was diluted in a 1:1 dilution ratio from 200 nM (see detailed results for actual concentrations tested) to obtain 5 concentrations in total. The dissociation phase was monitored for up to 600 s and triggered by switching from the sample solution to the running buffer. The surface was regenerated by washing with 10 mM glycine solution (pH 1.5) at a flow rate of 30 μL/min for 30 s. The difference in bulk refractive index was corrected by subtracting the responses obtained from the goat anti-human Fc surface. Blank injections (double reference) were also subtracted. To calculate the apparent KD and other kinetic parameters, the Langmuir 1:1 model was used. The association rate (Kₐ), dissociation rate (K_{d}), and binding affinity (KD) of the anti-GPC3 humanized antibodies with the human GPC3 protein are shown in the table, in which the antibody Y035 was used as a control. As shown in Table 17, the affinity of the 4 anti-GPC3 humanized antibodies to the human GPC3 protein was greater than 1E-8M.

TABLE 17
Binding affinity of anti-GPC3 humanized
antibodies to human GPC3 protein

| Antibody name | Ka (1/Ms) | Kd (1/s) | KD (M) |
|---|---|---|---|
| S001-SIM2 | 1.92E+05 | 3.91E−04 | 2.04E−09 |
| S001-SIM3 | 1.94E+05 | 4.11E−04 | 2.12E−09 |
| S001-SIM4 | 1.87E+05 | 3.67E−04 | 1.96E−09 |
| S001-SIM5 | 2.11E+05 | 2.25E−04 | 1.07E−09 |
| Y035 | 1.55E+05 | 2.73E−04 | 1.76E−09 |

### (B) Assay for Affinity of Anti-GPC3 Humanized Antibodies to Monkey GPC3-his Protein

The affinity of the anti-GPC3 humanized antibodies to the monkey GPC3-His protein was assayed according to the method described in Example 5 (A), in which the antibody Y035 was used as a control. As shown in Table 18, the affinity of the 4 anti-GPC3 humanized antibodies to the monkey GPC3 protein was greater than 1E-8M, among which the affinity of SIMS was greater than 1E-9M, reaching 5.41E-10M.

TABLE 18
Binding affinity of anti-GPC3 humanized
antibodies to monkey GPC3 protein

| Antibody name | Ka (1/Ms) | Kd (1/s) | KD (M) |
|---|---|---|---|
| S001-SIM2 | 2.76E+05 | 4.07E−04 | 1.47E−09 |
| S001-SIM3 | 3.38E+05 | 3.83E−04 | 1.13E−09 |
| S001-SIM4 | 3.08E+05 | 3.66E−04 | 1.19E−09 |
| S001-SIM5 | 3.75E+05 | 2.03E−04 | 5.41E−10 |
| Y035 | 2.47E+05 | 3.13E−04 | 1.27E−09 |

### Example 6: Analysis on Antigen-Binding Epitopes of Antibodies (Identification of Antigen-Binding Regions of Antibodies)

The mature GPC3 protein has a soluble N-terminal peptide of about 40 kD that can enter the blood and a membrane-bound C-terminal peptide of about 30 kD. The antibody Y035 recognizes a region (membrane-proximal region) that is near the cell membrane and that is at the C-terminus of the GPC3 protein, while the antibody T2-23 recognizes non-membrane-proximal regions. To identify whether antigen-binding epitopes of the anti-GPC3 humanized antibodies were located at the membrane proximal end, membrane proximal binding of the anti-GPC3 humanized antibodies was identified by coating the polypeptide GC3pep (membrane proximal end) of human GPC3 according to the ELISA method described in Example 1 (A). As shown in FIG. 12 and Table 19, the 4 anti-GPC3 humanized antibodies could recognize GC3pep, indicating that they are antibodies recognizing membrane-proximal epitopes.

TABLE 19
Assay results for binding reactions of anti-GPC3 humanized
antibodies with polypeptide GC3pep by ELISA

| | Antibody concentration (nM) | | | | | | | |
|---|---|---|---|---|---|---|---|---|
| | | | | | | | | Blank |
| Antibody | | | | | | | | con- |
| name | 100 | 10 | 1 | 0.1 | 0.01 | 0.001 | 0.0001 | trol |
| S001-SIM2 | 2.22 | 2.45 | 2.29 | 1.03 | 0.17 | 0.07 | 0.05 | 0.04 |
| S001-SIM3 | 2.21 | 2.38 | 2 | 0.93 | 0.16 | 0.06 | 0.47 | 0.05 |
| S001-SIM4 | 2.39 | 2.41 | 2.12 | 0.94 | 0.17 | 0.08 | 0.05 | 0.06 |
| S001-SIM5 | 2.4 | 2.43 | 1.51 | 0.85 | 0.18 | 0.17 | 0.11 | 0.42 |
| Y035 | 2.22 | 2.27 | 1.64 | 0.57 | 0.12 | 0.06 | 0.55 | 0.08 |
| T2-23 | 0.72 | 0.14 | 0.06 | 0.04 | 0.06 | 0.06 | 0.11 | 0.05 |
| hIgG1 | 0.06 | 0.04 | 0.04 | 0.04 | 0.05 | 0.05 | 0.05 | 0.05 |

The GPC3 humanized antibodies and use thereof provided in the present invention are described in detail above. The principle and implementations of the present invention are described with specific examples herein. The foregoing examples are merely intended to help understand the method and core idea of the present invention. It should be pointed out that those skilled in the art can further make several improvements and modifications to the present invention, without departing from the principle of the present invention; moreover, these improvements and modifications also fall within the protection scope of the claims of the present invention.

1. An antibody or an antigen-binding fragment specifically binding to GPC3, comprising a humanized heavy chain variable region and/or a humanized light chain variable region, wherein the heavy chain variable region comprises a human-derived heavy chain framework region, a complementarity determining region (CDR) 1 comprising an amino acid sequence set forth in SEQ ID NO: 15, a CDR2 comprising an amino acid sequence set forth in SEQ ID NO: 16, and a CDR3 comprising an amino acid sequence set forth in SEQ ID NO: 17; and the light chain variable region comprises a human-derived light chain framework region, a complementarity determining region (CDR) 1 comprising amino acid sequences set forth in SEQ ID NOs: 18 and 21, a CDR2 comprising an amino acid sequence set forth in SEQ ID NO: 19, and a CDR3 comprising an amino acid sequence set forth in SEQ ID NO: 20.

2. The antibody or the antigen-binding fragment according to claim 1, wherein the heavy chain framework region comprises framework regions HFR1, HFR2 and HFR3 of IGHV1-3*01 set forth in SEQ ID NO: 11, and a framework region HFR4 of IGHJ1*01 set forth in SEQ ID NO: 12; and the light chain framework region comprises framework regions LFR1, LFR2 and LFR3 of IGKV2-40*01 set forth in SEQ ID NO: 9, and a framework region LFR4 of IGKJ2*01 set forth in SEQ ID NO: 10, wherein the framework regions are determined according to a Kabat numbering scheme.

3. The antibody or the antigen-binding fragment according to claim 2, wherein the heavy chain framework region, numbered according to the Kabat numbering scheme, comprises a mutation of an amino acid residue at a position selected from positions 1, 44, 69, 71, 73, and 93, and preferably, the mutation comprises: Q1E, R71A, and A93T; Q1E, I69L, R71A, and A93T; or Q1E, R44G, I69L, R71A, T73K, and A93T.

4. The antibody or the antigen-binding fragment according to claim 2, wherein the light chain framework region, numbered according to the Kabat numbering scheme, comprises at most one mutation of an amino acid residue; preferably, the mutation is a mutation of an amino acid residue at a position selected from position 2; and preferably, the mutation is I2V.

5. The antibody or the antigen-binding fragment according to claim 1, wherein the antibody or the antigen-binding fragment comprises: (1) a heavy chain variable region set forth in any one of SEQ ID NOs: 4-6, or a sequence having at least 80%, 85%, 90%, 91%, 92%, 93%, 94%, 95%, 96%, 97%, 98%, 99%, or 100% identity to the sequence set forth in any one of SEQ ID NOs: 4-6; or a sequence having at most 20, 19, 18, 17, 16, 15, 14, 13, 12, 11, 10, 9, 8, 7, 6, 5, 4, 3, 2, or 1 mutation compared with the sequence set forth in any one of SEQ ID NOs: 4-6; the mutation can be selected from an insertion, a deletion and/or a substitution, and preferably, the substitution is a conservative amino acid substitution;
and/or, (2) a light chain variable region set forth in any one of SEQ ID NOs: 7-8 and 13-14, or a sequence having at least 80%, 85%, 90%, 91%, 92%, 93%, 94%, 95%, 96%, 97%, 98%, 99%, or 100% identity to the sequence set forth in any one of SEQ ID NOs: 7-8 and 13-14; or a sequence having at most 20, 19, 18, 17, 16, 15, 14, 13, 12, 11, 10, 9, 8, 7, 6, 5, 4, 3, 2, or 1 mutation compared with the sequence set forth in any one of SEQ ID NOs: 7-8 and 13-14; the mutation can be selected from an insertion, a deletion, and/or a substitution, and preferably, the substitution is a conservative amino acid substitution.

6. The antibody or the antigen-binding fragment according to claim 5, wherein the antibody or the antigen-binding fragment comprises:
(1) a heavy chain variable region having a sequence set forth in SEQ ID NO: 4, 5, or 6, and a light chain variable region having a sequence set forth in SEQ ID NO: 14; or
(2) a heavy chain variable region having a sequence set forth in SEQ ID NO: 5, and a light chain variable region having a sequence set forth in SEQ ID NO: 7; or
(3) a heavy chain variable region and a light chain variable region having sequences having at least 70%, 75%, 80%, 85%, 90%, 95%, 96%, 97%, 98%, 99% or more identity to the sequences set forth in (1) and (2) above, respectively.

7. The antibody or the antigen-binding fragment according to claim 1, wherein the antibody or the antigen-binding fragment comprises a sequence having at least 80%, 85%, 90%, 91%, 92%, 93%, 94%, 95%, 96%, 97%, 98%, 99%, or 100% identity to the CDR1, the CDR2, and/or the CDR3; or a sequence having 1, 2, 3 or more amino acid insertions, deletions and/or substitutions compared with the CDR1, the CDR2, and/or the CDR3, and preferably, the substitutions are conservative amino acid substitutions.

8. The antibody or the antigen-binding fragment according to claim 1, wherein the antibody or the antigen-binding fragment specifically binds to a human and/or monkey GPC3 protein; and preferably, the antibody or the antigen-binding fragment binds to the human and/or monkey GPC3 with a dissociation constant (KD) not greater than 1.00E-7 M, 1.00E-8 M, 2.00E-8 M, 3.00E-8 M, 4.00E-8 M, 5.00E-8 M, 6.00E-8 M, 7.00E-8 M, 8.00E-8 M, 9.00E-8 M, 1.00E-9 M, 2.00E-9 M, 3.00E-9 M, 4.00E-9 M, 5.00E-9 M, 6.00E-9 M, 7.00E-9 M, 8.00E-9 M, 9.00E-9 M, or 6.00E-10 M.

9. The antibody or the antigen-binding fragment according to claim 1, wherein the antibody or the antigen-binding fragment is selected from full-length antibodies, VH single domain antibodies, Fab fragments, Fab′ fragments, F(ab)′2 fragments, Fd fragments, Fv fragments, complementarity determining region (CDR) fragments, single chain variable fragments (scFvs), scFv2, disulfide stabilized variable fragments (dsFvs), domain antibodies, bivalent single chain antibodies, single chain phage antibodies, bispecific diabodies, triabodies, tetrabodies, and minimal recognition units of antibodies.

10. An immunoconjugate, wherein the immunoconjugate comprises the antibody or the antigen-binding fragment according to claim 1 and an effector molecule; and preferably, the effector molecule is linked to the antibody or the antigen-binding fragment; optionally, the effector molecule comprises a therapeutic agent or a marker; and optionally, the therapeutic agent is selected from a drug, a toxin, a radioisotope, a chemotherapeutic drug, and an immunomodulator; or
the immunoconjugate further comprises a linker for conjugating the effector molecule to the antibody, and the linker comprises but is not limited to hydrazones, thioethers, esters, disulfides, and peptide-containing linkers.

11. (canceled)

12. (canceled)

13. A chimeric antigen receptor (CAR), wherein the CAR comprises an extracellular antigen-binding domain, a transmembrane domain, and an intracellular signaling domain, and the extracellular antigen-binding domain comprises the antibody or the antigen-binding fragment according to claim 1.

14. An immunocompetent cell, wherein the immunocompetent cell expresses the chimeric antigen receptor according to claim 13 or comprises a nucleic acid molecule encoding the chimeric antigen receptor according to claim 13; and preferably, the immunocompetent cell is selected from: a T cell, a natural killer cell (NK cell), a natural killer T cell (NKT cell), a double negative T cell (DNT cell), a monocyte, a macrophage, a dendritic cell, and a mast cell, and the T cell is preferably selected from a cytotoxic T cell, a regulatory T cell, and a helper T cell.

15. A multispecific molecule, wherein the multispecific molecule comprises the antibody or the antigen-binding fragment according to claim 1; and preferably, the multispecific molecule further comprises an antibody or an antigen-binding fragment specifically binding to an antigen other than GPC3 or binding to a GPC3 epitope different from the antibody or the antigen-binding fragment according to claim 1; optionally, the antigen other than GPC3 is an antigen on surface of a T cell, a B cell, a natural killer cell, a dendritic cell, a macrophage, a monocyte, or a neutrophil; and preferably, the antigen other than GPC3 is selected from: CD3, CD3γ, CD3δ, CD3ε, CD3ζ, CD16, CD16A, CD32B, PD-1, PD-2, PD-L1, VEGF, NKG2D, CD19, CD20, CD40, CD47, 4-1BB, CD137, EGFR, EGFRvIII, TNF-alpha, CD33, HER2, HER3, HAS, CD5, CD27, EphA2, EpCAM, MUC1, MUC16, CEA, Claudin18.2, a folate receptor, Claudin6, WT1, NY-ESO-1, MAGE3, ASGPR1, and CDH16; or
the multispecific molecule is a tandem scFv, a bifunctional antibody (Db), a single chain bifunctional antibody (scDb), a dual affinity retargeting (DART) antibody, F(ab′)2, a dual variable domain (DVD) antibody, a knobs-into-holes (KiH) antibody, a dock-and-lock (DNL) antibody, a chemically cross-linked antibody, a heteropolyantibody or a heteroconjugate antibody.

16. (canceled)

17. (canceled)

18. An isolated nucleic acid molecule, wherein the nucleic acid molecule encodes the antibody or the antigen-binding fragment according to claim 1.

19. A vector, wherein the expression vector comprises the nucleic acid molecule according to claim 18.

20. A host cell, wherein the host cell comprises the nucleic acid molecule according to claim 18, and preferably, the host cell is a prokaryotic cell or a eukaryotic cell, comprising a bacteria (E. coli), a fungus (yeast), an insect cell, or a mammalian cell (CHO cell line or 293T cell line).

21. (canceled)

22. (canceled)

23. A pharmaceutical composition, comprising a therapeutically effective amount of the antibody or the antigen-binding fragment according to claim 1, and a pharmaceutically acceptable carrier.

24. (canceled)

25. A method for treating a subject suffering from a GPC3-mediated tumor, comprising: administering to the subject a therapeutically effective amount of the antibody or the antigen-binding fragment according to claim 1, wherein the tumor is preferably selected from diseases such as hepatocellular carcinoma, melanoma, ovarian clear cell carcinoma, hepatoblastoma, neuroblastoma, Wilms' tumor, small cell lung cancer, lung adenocarcinoma, gastric cancer, colon cancer, rectal cancer, cervical cancer, breast cancer, ovarian cancer, skin cancer, lymphoma, prostate cancer, pancreatic cancer, renal cancer, esophageal cancer, thyroid cancer, testicular cancer, bladder cancer, bronchogenic carcinoma, nasopharyngeal cancer, head and neck cancer, endometrial cancer, brain cancer, bone cancer, leukemia, malignant mesothelioma, and liposarcoma; and preferably, hepatocellular carcinoma.

26. (canceled)

27. A kit, comprising the antibody or the antigen-binding fragment according to claim 1.

28. (canceled)

29. A method for detecting GPC3 expression in a biological sample, comprising: exposing a sample from a subject to the antibody or the antigen-binding fragment according to claim 1, and detecting binding of the antibody or the antigen-binding fragment to the sample.


SEQUENCE LISTING (27 sequences)

SEQ ID NO: 1 - protein, 475 aa, Artificial Sequence
  Synthetic polypeptide
DIVMTQTPLSLPVITGEPASISCRSSQSLVHSNGNTYLQWYLQKPGQSPQLLIYKVSNRFSGVPDRFSGSGSGTDFTLKISRVEAEDVGVYYCSQSIYVPYTFGQGTKLEIKRGGGGSGGGGSGGGGSEVQLVQSGAEVKKPGASVKVSCKASGYTFSDYEMHWVRQAPGQGLEWMGAIHPGSGDTAYNQRFKGRVTITADKSTSTAYMELSSLRSEDTAVYYCARFYSYAYWGQGTLVTVSAEPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 479 aa, Artificial Sequence
  Synthetic polypeptide
QSALTQPPSASGSPGQSVTISCTGTSSDVGGYNYVSWYQQYPGKAPKLLIYGNSNRPSGVPDRFSGSKSGTSASLAITGLQAEDGADYYCQSYDSSLRVVFGGGTKVTVLGGGGGSGGGGSGGGGSQVQLQESGGGLVQPGRSLRLSCAASGFTFSSYAMHWVRQAPGKGLEWVSAISSSGRSTYYADSVEGRFTISRDNSKNTLYLQMNSLRAEDTAVYYCAKDRRGSHADALNVWGQGTLVTVSSEPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 40 aa, Artificial Sequence
  Synthetic polypeptide
AELAYDLDVDDAPGNSQQATPKDNEISTFHNLGNVHSPLK

SEQ ID NO: 4 - protein, 115 aa, Artificial Sequence
  Synthetic polypeptide
EVQLVQSGAEVKKPGASVKVSCKASGYTFTDYEMHWVRQAPGQRLEWMGALDPKTGDTAYSQKFKGRVTITADTSASTAYMELSSLRSEDTAVYYCTRFYSYTYWGQGTLVTVSS

SEQ ID NO: 5 - protein, 115 aa, Artificial Sequence
  Synthetic polypeptide
EVQLVQSGAEVKKPGASVKVSCKASGYTFTDYEMHWVRQAPGQRLEWMGALDPKTGDTAYSQKFKGRVTLTADTSASTAYMELSSLRSEDTAVYYCTRFYSYTYWGQGTLVTVSS

SEQ ID NO: 6 - protein, 115 aa, Artificial Sequence
  Synthetic polypeptide
EVQLVQSGAEVKKPGASVKVSCKASGYTFTDYEMHWVRQAPGQGLEWMGALDPKTGDTAYSQKFKGRVTLTADKSASTAYMELSSLRSEDTAVYYCTRFYSYTYWGQGTLVTVSS

SEQ ID NO: 7 - protein, 112 aa, Artificial Sequence
  Synthetic polypeptide
DIVMTQTPLSLPVTPGEPASISCRSSQSLVHSNGNTYLHWYLQKPGQSPQLLIYKVSNRFSGVPDRFSGSGSGTDFTLKISRVEAEDVGVYYCSQNTHVPPTFGQGTKLEIK

SEQ ID NO: 8 - protein, 112 aa, Artificial Sequence
  Synthetic polypeptide
DVVMTQTPLSLPVTPGEPASISCRSSQSLVHSNGNTYLHWYLQKPGQSPQLLIYKVSNRFSGVPDRFSGSGSGTDFTLKISRVEAEDVGVYYCSQNTHVPPTFGQGTKLEIK

SEQ ID NO: 9 - protein, 101 aa, Artificial Sequence
  Synthetic polypeptide
DIVMTQTPLSLPVTPGEPASISCRSSQSLLDSDDGNTYLDWYLQKPGQSPQLLIYTLSYRASGVPDRFSGSGSGTDFTLKISRVEAEDVGVYYCMQRIEFP

SEQ ID NO: 10 - protein, 10 aa, Artificial Sequence
  Synthetic polypeptide
FGQGTKLEIK

SEQ ID NO: 11 - protein, 98 aa, Artificial Sequence
  Synthetic polypeptide
QVQLVQSGAEVKKPGASVKVSCKASGYTFTSYAMHWVRQAPGQRLEWMGWINAGNGNTKYSQKFQGRVTITRDTSASTAYMELSSLRSEDTAVYYCAR

SEQ ID NO: 12 - protein, 11 aa, Artificial Sequence
  Synthetic polypeptide
WGQGTLVTVSS

SEQ ID NO: 13 - protein, 112 aa, Artificial Sequence
  Synthetic polypeptide
DIVMTQTPLSLPVTPGEPASISCRSSQSLVHSNANTYLHWYLQKPGQSPQLLIYKVSNRFSGVPDRFSGSGSGTDFTLKISRVEAEDVGVYYCSQNTHVPPTFGQGTKLEIK

SEQ ID NO: 14 - protein, 112 aa, Artificial Sequence
  Synthetic polypeptide
DVVMTQTPLSLPVTPGEPASISCRSSQSLVHSNANTYLHWYLQKPGQSPQLLIYKVSNRFSGVPDRFSGSGSGTDFTLKISRVEAEDVGVYYCSQNTHVPPTFGQGTKLEIK

SEQ ID NO: 15 - protein, 5 aa, Artificial Sequence
  Synthetic polypeptide
DYEMH

SEQ ID NO: 16 - protein, 17 aa, Artificial Sequence
  Synthetic polypeptide
ALDPKTGDTAYSQKFKG

SEQ ID NO: 17 - protein, 6 aa, Artificial Sequence
  Synthetic polypeptide
FYSYTY

SEQ ID NO: 18 - protein, 16 aa, Artificial Sequence
  Synthetic polypeptide
RSSQSLVHSNGNTYLH

SEQ ID NO: 19 - protein, 7 aa, Artificial Sequence
  Synthetic polypeptide
KVSNRFS

SEQ ID NO: 20 - protein, 9 aa, Artificial Sequence
  Synthetic polypeptide
SQNTHVPPT

SEQ ID NO: 21 - protein, 16 aa, Artificial Sequence
  Synthetic polypeptide
RSSQSLVHSNANTYLH

SEQ ID NO: 22 - protein, 23 aa, Artificial Sequence
  Synthetic polypeptide
DIVMTQTPLSLPVTPGEPASISC

SEQ ID NO: 23 - protein, 15 aa, Artificial Sequence
  Synthetic polypeptide
WYLQKPGQSPQLLIY

SEQ ID NO: 24 - protein, 32 aa, Artificial Sequence
  Synthetic polypeptide
GVPDRFSGSGSGTDFTLKISRVEAEDVGVYYC

SEQ ID NO: 25 - protein, 30 aa, Artificial Sequence
  Synthetic polypeptide
QVQLVQSGAEVKKPGASVKVSCKASGYTFT

SEQ ID NO: 26 - protein, 14 aa, Artificial Sequence
  Synthetic polypeptide
WVRQAPGQRLEWMG

SEQ ID NO: 27 - protein, 32 aa, Artificial Sequence
  Synthetic polypeptide
RVTITRDTSASTAYMELSSLRSEDTAVYYCAR